(19) United States
(12) Patent Application Publication
(10) Pub. No.: US 2026/0194535 A1
(43) Pub. Date: Jul. 9, 2026

(54) A METHOD OF EARLY PREDICTION OF RISK OF PRE-TERM PRE-ECLAMPSIA IN SPECIFIC POPULATION COHORTS

(71) Applicant: METABOLOMIC DIAGNOSTICS LIMITED, Inchera, Co. Cork (IE)

(72) Inventors: Robin TUYTTEN, Midleton, Co. Cork (IE); Paloma MUNIZ ORTEA, Lugones (ES); Gregoire THOMAS, Lokeren (BE)

(73) Assignee: METABOLOMIC DIAGNOSTICS LIMITED, Inchera, Co. Cork (IE)

(21) Appl. No.: 19/128,721

(22) PCT Filed: Oct. 25, 2023
(86) PCT No.: PCT/EP2023/079741

§ 371 Date: May 9, 2025

(30) Foreign Application Priority Data
Nov. 10, 2022 (EP) 22206717.5

(51) Int. Cl. G01N 33/68

(52) U.S. Cl. CPC G01N 33/689; G01N 2800/368

(57) ABSTRACT

Methods of detecting pre-term pre-eclampsia in specific sub-cohorts of pregnant women are described. The methods employ metabolite biomarkers and ratios of metabolite biomarkers. Systems for early calculation of risk of pre-term pre-eclampsia in specific sub-cohorts of pregnant women are also described.

### FIELD OF THE INVENTION

The present invention relates to a method of early prediction of risk of pre-term pre-eclampsia in specific population cohorts.

### BACKGROUND TO THE INVENTION

Preeclampsia, which complicates 2 to 4% of pregnancies globally, is progressive, unpredictable, and serious. Today, preeclampsia is still associated with approximately 46,000 maternal deaths and approximately 500,000 fetal and new-born deaths annually. It is now widely accepted that preeclampsia is not a single disorder but a syndrome with distinct etiologies. Yet, thus far, the increased understanding of pathophysiology has not contributed significantly to bettering prediction nor to expanding prevention or treatment options. To explain this lack of progress, it has been hypothesized that the maternal syndrome develops through distinct pathophysiological pathways. If confirmed, there is a need to recognize different subtypes of preeclampsia to yield more clinical utility. Recent data from Than et al., suggest that there are distinct maternal and placental disease pathways and that their interaction determines the clinical presentation of preeclampsia. With activation of maternal disease pathways detected in advance of placental dysfunction, their data also pointed to pre-existing, possibly sub-clinical, maternal risk profiles.

Current benchmark prediction models for preeclampsia risk combine maternal risk factors, Doppler velocimetry of the uterine arteries, mean arterial pressure and the blood levels of the proteins placental growth factor (PIGF) and pregnancy-associated plasma protein-A (PAPP-A), whereby the biophysical and biochemical data are transformed into multiples of the median (MoM) values using population- and site-specific models. The detection rate of these models for identifying patients at risk for preterm preeclampsia, i.e. preeclampsia leading to delivery before 37 weeks of gestation, already enables preventive strategies 10. About 30% of all pre-eclampsia is preterm preeclampsia.

For prevention of term preeclampsia, other interventions may be more relevant, e.g. like metformin. The latter has been shown prevent preeclampsia in obese pregnant women. Yet, to further improve detection rates, preeclampsia risk models will need to consider additional biomarkers, and preferably these biomarkers associate with specific preeclampsia risk profiles or patient phenotypes. Accounting for different maternal risk profiles may allow for the formulation of improved, yet more complex, risk prediction models. From the moment different pathophysiological pathways for preeclampsia can be delineated the alluring possibility for more targeted, personalised pharmaceutical interventions, like the stratification to aspirin or metformin prophylaxis can be realised.

It is an objective of the invention to overcome at least one of the above-referenced problems.

### SUMMARY OF THE INVENTION

In a first aspect, the invention provides a method of early prediction of risk of pre-term pre-eclampsia in a pregnant human subject having a BMI of less than 25, the method comprising the steps of:
providing an abundance of ornithine in a biological sample obtained from the subject at an early stage of pregnancy;
comparing the abundance of ornithine in the subject to a reference abundance; and
providing an estimation of the risk of pre-term pre-eclampsia in the subject based on the comparison.

In a further aspect, the invention provides a system to calculate risk of pre-term pre-eclampsia in a pregnant human subject having a BMI of less than 25, the system a processor configured to:
receive as an input an abundance of a metabolite biomarker of Table 5 in a biological sample obtained from the subject at an early stage of pregnancy; compare the abundance of the metabolite biomarker in the subject to a reference abundance;
calculate the risk of pre-term pre-eclampsia in the subject based on the comparison; and provide an output of the risk.

In any embodiment, the metabolite biomarker is ornithine.

In any embodiment, the reference abundance is a reference abundance of ornithine.

In any embodiment:
the reference abundance of the metabolite biomarker is the abundance of the metabolite biomarker in a negative control, wherein a reduced abundance of the metabolite biomarker relative to the reference abundance correlates with risk of pre-term pre-eclampsia; or
the reference abundance of the metabolite biomarker is the abundance of the metabolite biomarker in a positive control, wherein a similar abundance of the metabolite biomarker relative to the reference abundance correlates with risk of pre-term pre-eclampsia.

In any embodiment:
the reference abundance is an abundance of a second metabolite biomarker in a biological sample obtained from the subject at an early stage of pregnancy, in which the second metabolite biomarker is selected from threonine, Asymmetric dimethylarginine (ADMA), 1-Oleoyl-2-hydroxy-sn-glycero-3-phosphocholine, 1-Palmitoyl-2-hydroxy-sn-glycero-3-phosphocholine, alanine and glutamine; and the comparing step comprises calculating a ratio of the abundance of the (first) metabolite biomarker and the abundance of one of the second metabolite biomarkers.

In any embodiment:
the ratio is second metabolite biomarker/ornithine; and
the second metabolite biomarker is selected from threonine, Asymmetric dimethylarginine (ADMA), 1-Oleoyl-2-hydroxy-sn-glycero-3-phosphocholine, 1-Palmitoyl-2-hydroxy-sn-glycero-3-phosphocholine, alanine,
wherein a ratio of greater than 1 (for example at least 1.1, 1.5 or 1.2) correlates with risk of pre-term pre-eclampsia.

In any embodiment:
the ratio is second metabolite biomarker/ornithine; and the second metabolite biomarker is glutamine,
wherein a ratio of less than 1 (for example less than 0.95 or 0.9) correlates with risk of pre-term pre-eclampsia.

In any embodiment, the ratio is selected from the following metabolite ratio's:
Citrulline/1-Oleoyl-2-hydroxy-sn-glycero-3-phosphocholine;
1-(9Z-Octadecenoyl)-sn-glycero-3-phospho-L-serine/1-Oleoyl-2-hydroxy-sn-glycero-3-phosphocholine;
Citrulline/1-Palmitoyl-2-hydroxy-sn-glycero-3-phosphocholine; and Citrulline/h-Arginine,
   wherein a ratio of less than 1 (for example less than 0.95 or 0.9 or 0.8) correlates with risk of pre-term pre-eclampsia.

In any embodiment, the ratio is selected from the following metabolite ratio's:
25-Hydroxyvitamin D3/Citrulline; and
1-Oleoyl-2-hydroxy-sn-glycero-3-phosphocholine/1-(1Z-Octadecenyl)-2-oleoyl-sn-glycero-3-phosphocholine,
   wherein a ratio of greater than 1 (for example at least 1.1, 1.5 or 1.2) correlates with risk of pre-term pre-eclampsia.

In a further aspect, the invention provides method of early prediction of risk of pre-term pre-eclampsia in a pregnant human subject having a BMI of 30 or greater, the method comprising the steps of:
providing an abundance of a metabolite biomarker in a biological sample obtained from the subject at an early stage of pregnancy;
comparing the abundance of the biomarker in the subject to a reference abundance; and
providing an estimation of the risk of pre-term pre-eclampsia in the subject based on the comparison,
in which the metabolite biomarker is selected from 2-Hydroxy-(2/3)-methylbutyric acid, Dodecanoylcarnitine, Alanine, Decanoylcarnitine and Symmetric dimethylarginine.

In a further aspect, the invention provides a system to calculate risk of pre-term pre-eclampsia in a pregnant human subject having a BMI of 30 or greater, the system comprising a processor configured to:
receive as an input an abundance of a metabolite biomarker in a biological sample obtained from the subject at an early stage of pregnancy;
compare the abundance of the biomarker in the subject to a reference abundance;
calculate the risk of pre-term pre-eclampsia in the subject based on the comparison; and
provide an output of the risk,
in which the metabolite biomarker is selected from 2-Hydroxy-(2/3)-methylbutyric acid, Dodecanoylcarnitine, Alanine, Decanoylcarnitine and Symmetric dimethylarginine.

In any embodiment, the reference abundance is a reference abundance of the metabolite biomarker.

In any embodiment:
the reference abundance of the metabolite biomarker is the abundance of the metabolite biomarker in a negative control, wherein an increased abundance of the metabolite biomarker relative to the reference abundance correlates with risk of pre-term pre-eclampsia; or
the reference abundance of the metabolite biomarker is the abundance of the metabolite biomarker in a positive control, wherein a similar abundance of the metabolite biomarker relative to the reference abundance correlates with risk of pre-term pre-eclampsia.

In any embodiment, the reference abundance is an abundance of a second metabolite biomarker of Table 8 in a biological sample obtained from the subject at an early stage of pregnancy, wherein the comparing step comprises calculating (optionally by the processor) a ratio of the abundance of the (first) metabolite biomarker and the abundance of one of the second metabolite biomarkers.

In any embodiment, the metabolite ratio is selected from:
2-Hydroxy-(2/3)-methylbutyric acid/1-(1Z-Octadecenyl)-2-oleoyl-sn-glycero-3-phosphocholine;
2-Hydroxy-(2/3)-methylbutyric acid/Glutamine;
2-Hydroxybutyric acid/1-(1Z-Octadecenyl)-2-oleoyl-sn-glycero-3-phosphocholine;
Dodecanoylcarnitine/Myristic acid
Symmetric dimethylarginine/1-(1Z-Octadecenyl)-2-oleoyl-sn-glycero-3-phosphocholine; Methionine/Glutamine;
Dodecanoylcarnitine/1-(1Z-Octadecenyl)-2-oleoyl-sn-glycero-3-phosphocholine;
Dodecanoylcarnitine/Palmitic acid;
Methionine/1-(1Z-Octadecenyl)-2-oleoyl-sn-glycero-3-phosphocholine;
Alanine/Glutamine;
Symmetric dimethylarginine/Glutamine;
Stearoylcarnitine/1-(1Z-Octadecenyl)-2-oleoyl-sn-glycero-3-phosphocholine);
Arginine/1-(1Z-Octadecenyl)-2-oleoyl-sn-glycero-3-phosphocholine;
Asymmetric dimethylarginine/1-(1Z-Octadecenyl)-2-oleoyl-sn-glycero-3-phosphocholine;
Decanoylcarnitine/1-(1Z-Octadecenyl)-2-oleoyl-sn-glycero-3-phosphocholine;
2-Hydroxybutyric acid/Glutamine;
Proline/1-(1Z-Octadecenyl)-2-oleoyl-sn-glycero-3-phosphocholine;
Palmitoylcarnitine/1-(1Z-Octadecenyl)-2-oleoyl-sn-glycero-3-phosphocholine;
Decanoylcarnitine)/Myristic acid;
NG-Monomethyl-L-arginine/1-(1Z-Octadecenyl)-2-oleoyl-sn-glycero-3-phosphocholine; and
Arginine/Glutamine,
wherein a ratio of greater than 1 (for example at least 1.1, 1.2, 1.3 or 1.4) correlates with risk of pre-term pre-eclampsia.

In any embodiment:
the metabolite biomarker is 2-Hydroxy-(2/3)-methylbutyric acid;
the reference abundance is an abundance of a second metabolite biomarker in a biological sample obtained from the subject at an early stage of pregnancy, in which the second metabolite is selected from 1-(1Z-Octadecenyl)-2-oleoyl-sn-glycero-3-phosphocholine and glutamine; and
the comparing step comprises calculating a ratio of the abundance of 2-Hydroxy-(2/3)-methylbutyric acid and the abundance of one of the second metabolite biomarkers.

In any embodiment:
the ratio is selected from:
2-Hydroxy-(2/3)-methylbutyric acid/1-(1Z-Octadecenyl)-2-oleoyl-sn-glycero-3-phosphocholine; and
2-Hydroxy-(2/3)-methylbutyric acid/glutamine,
wherein a ratio of greater than 1 (for example at least 1.2 or 1.3) correlates with risk of pre-term pre-eclampsia.

In any embodiment:
the metabolite biomarker is 2-Hydroxybutyric acid; and
the comparing step comprises calculating a ratio of the abundance of 2-Hydroxybutyric acid and the abundance of 1-(1Z-Octadecenyl)-2-oleoyl-sn-glycero-3-phosphocholine.

In any embodiment:
the ratio is 2-Hydroxybutyric acid/1-(1Z-Octadecenyl)-2-oleoyl-sn-glycero-3-phosphocholine; and
wherein a ratio of greater than 1 (for example at least 1.1 or 1.2) correlates with risk of pre-term pre-eclampsia.

In any embodiment:
the metabolite biomarker is Dodecanoylcarnitine; and
the comparing step comprises calculating a ratio of the abundance of Dodecanoylcarnitine and the abundance of Myristic acid.

In any embodiment:
the ratio is Dodecanoylcarnitine/Myristic acid; and
wherein a ratio of greater than 1 (for example at least 1.2.1.3 or 1.4) correlates with risk of pre-term pre-eclampsia.

In any embodiment, the ratio is selected from:

In a further aspect, the invention provides a method of early prediction of risk of pre-term pre-eclampsia in a pregnant human subject of white racial origin, the method comprising the steps of:
providing an abundance of a metabolite biomarker selected from 25-Hydroxyvitamin D3 or 1-Palmitoyl-2-hydroxy-sn-glycero-3-phosphocholine in a biological sample obtained from the subject at an early stage of pregnancy;
comparing the abundance of the metabolite biomarker in the subject to a reference abundance of the same metabolite biomarker; and
providing an estimation of the risk of pre-term pre-eclampsia in the subject based on the comparison.

In a further aspect, the invention provides a system to calculate risk of pre-term pre-eclampsia in a pregnant human subject of white racial origin, the system comprising
a processor configured to:
receive as an input an abundance of a metabolite biomarker selected from 25-Hydroxyvitamin D3 or 1-Palmitoyl-2-hydroxy-sn-glycero-3-phosphocholine in a biological sample obtained from the subject at an early stage of pregnancy; compare the abundance of the metabolite biomarker in the subject to a reference abundance of the same metabolite biomarker;
calculate the risk of pre-term pre-eclampsia in the subject based on the comparison; and
provide an output of the risk.

In any embodiment:
an increased abundance of the metabolite biomarker relative to the reference abundance of the metabolite biomarker in a negative control correlates with risk of pre-term pre-eclampsia; or
a similar abundance of the metabolite biomarker relative to the reference abundance of the metabolite biomarker in a positive control correlates with risk of pre-term pre-eclampsia.

In a further aspect, the invention provides a method of early prediction of risk of pre-term pre-eclampsia in a pregnant human subject of white racial origin, the method comprising the steps of:
providing an input an abundance of a plurality of biomarkers from Table 12 in a biological sample obtained from the subject at an early stage of pregnancy; calculating a ratio of two of the plurality of metabolite biomarker; and providing an estimation of the risk of pre-term pre-eclampsia in the subject based on the calculated ratio.

In any embodiment, the ratio of two of the plurality of metabolite biomarkers is a metabolite ratio of Table 12.

In any embodiment, the method comprises providing an abundance of a first metabolite biomarker selected from
2-Hydroxybutyric acid, 1-Palmitoyl-2-hydroxy-sn-glycero-3-phosphocholine and methionine,
and a second metabolite biomarker selected from 8,11,14 Eicosatrienoic acid, Palmitic acid, Glutamine, Leucine, Myristic acid, Arachidonic acid, Acetylcarnitine, 2-Hydroxy-(2/3)-methylbutyric acid, 3-Hydroxybutyric acid, Eicosapentaenoic acid, and Lactic acid;
calculating a ratio of a first metabolite biomarker to a second metabolite biomarker; and
providing an estimation of the risk of pre-term pre-eclampsia in the subject based on the comparison.

In a further aspect, the invention provides system to calculate risk of pre-term pre-eclampsia in a pregnant human subject of white racial origin, the system comprising a processor configured to:
receive as an input an abundance of a plurality of biomarkers from Table 12 in a biological sample obtained from the subject at an early stage of pregnancy;
calculate a ratio of two of the plurality of metabolite biomarker;
calculate the risk of pre-term pre-eclampsia in the subject based on the calculated ratio of the first and second metabolite biomarkers; and
provide an output of the risk.

In any embodiment, the processor is configured to:
receive as inputs an abundance of a first metabolite biomarker selected from 2-Hydroxybutyric acid, 1-Palmitoyl-2-hydroxy-sn-glycero-3-phosphocholine and methionine,
and a second metabolite biomarker selected from
8,11,14 Eicosatrienoic acid, Palmitic acid, Glutamine, Leucine, Myristic acid,
Arachidonic acid, Acetylcarnitine, 2-Hydroxy-(2/3)-methylbutyric acid, 3-Hydroxybutyric acid, Eicosapentaenoic acid, and Lactic acid;
calculate a ratio of a first metabolite biomarker to a second metabolite biomarker;
calculate the risk of pre-term pre-eclampsia in the subject based on the calculated ratio of the first and second metabolite biomarkers; and
provide an output of the risk.

In a further aspect, the invention provides a method of early prediction of risk of pre-term pre-eclampsia in a pregnant human subject of black racial origin, the method comprising the steps of:
providing an abundance of Ergothioneine in a biological sample obtained from the subject at an early stage of pregnancy;
comparing the abundance of Ergothioneine in the subject to a reference abundance of Ergothioneine or to an abundance of Cotinine in a biological sample obtained from the subject at an early stage of pregnancy; and
providing an estimation of the risk of pre-term pre-eclampsia in the subject based on the comparison.

In a further aspect, the invention provides a system to calculate risk of pre-term pre-eclampsia in a pregnant human subject of black racial origin, the system comprising a processor configured to:
receive as an input an abundance of Ergothioneine and optionally Cotinine in a biological sample obtained from the subject at an early stage of pregnancy;
compare the abundance of Ergothioneine in the subject to a reference abundance of Ergothioneine or to an abundance of Cotinine in a biological sample obtained from the subject at an early stage of pregnancy; and calculate the risk of pre-term pre-eclampsia in the subject based on the comparison; and
provide an output of the risk.

In any embodiment:
an increased abundance of Ergothioneine relative to the reference abundance of Ergothioneine in a negative control correlates with risk of pre-term pre-eclampsia; or
a similar abundance of Ergothioneine relative to the reference abundance of Ergothioneine in a positive control correlates with risk of pre-term pre-eclampsia.

In any embodiment, a ratio of Ergothioneine/Cotinine of less than 1 correlates with risk of pre-term pre-eclampsia.

In any embodiment, a ratio of Cotinine/Ergothioneine of greater than 1 correlates with risk of pre-term pre-eclampsia.

In a further aspect, the invention provides a method of calculating risk of pre-term pre-eclampsia in a pregnant human subject, the method comprising the steps of: providing an abundance of a plurality of biomarkers from Table 4 in a biological sample obtained from the subject at an early stage of pregnancy;
calculating a ratio of two of the plurality of metabolite biomarker selected from:
25-Hydroxyvitamin D3 and 1-(1Z-Octadecenyl)-2-oleoyl-sn-glycero-3-phosphocholine
2-Hydroxy-(2/3)-methylbutyric acid and Ornithine;
2-Hydroxy-(2/3)-methylbutyric acid and 1-(1Z-Octadecenyl)-2-oleoyl-sn-glycero-3-phosphocholine;
25-Hydroxyvitamin D3 and Glutamine;
2-Hydroxy-(2/3)-methylbutyric acid and Glutamine;
2-Hydroxybutyric acid and 1-(1Z-Octadecenyl)-2-oleoyl-sn-glycero-3-phosphocholine;
Dodecanoylcarnitine) and Myristic acid;
Dodecanoylcarnitine and Bilirubin;
Alanine and Glutamine;
Alanine and 1-(1Z-Octadecenyl)-2-oleoyl-sn-glycero-3-phosphocholine);
2-Hydroxybutyric acid and Bilirubin;
Bilirubin and 2-Hydroxy-(2/3)-methylbutyric acid;
25-Hydroxyvitamin D3 and Ornithine;
2-Hydroxybutyric acid and Cotinine;
Cotinine and 2-Hydroxy-(2/3)-methylbutyric acid; and
Symmetric dimethylarginine and 1-(1Z-Octadecenyl)-2-oleoyl-sn-glycero-3-phosphocholine and
   estimating the risk of pre-term pre-eclampsia in the subject based on the calculated ratio of the first and second metabolite biomarkers. The ratio value may be calculated as A/B or B/A, for example 25(OH)D3/PC(18:1/18:1) or PC(18:1/18:1)/25(OH)D3, and then compared with Table 4 to determine risk. For example, in the case of 25(OH)D3 and PC(18:1/18:1), a ratio of 25(OH)D3/PC(18:1/18:1) of greater than 1 correlates whereas a ratio of PC(18:1/18:1)/25(OH)D3 of less than 1 correlates with risk.

In any embodiment, the ratio is selected from:
25-Hydroxyvitamin D3/1-(1Z-Octadecenyl)-2-oleoyl-sn-glycero-3-phosphocholine
2-Hydroxy-(2/3)-methylbutyric acid/Ornithine;
2-Hydroxy-(2/3)-methylbutyric acid/1-(1Z-Octadecenyl)-2-oleoyl-sn-glycero-3-phosphocholine;
25-hydroxyvitamin D3/Glutamine;
2-Hydroxy-(2/3)-methylbutyric acid/Glutamine;
2-Hydroxybutyric acid/1-(1Z-Octadecenyl)-2-oleoyl-sn-glycero-3-phosphocholine;
Dodecanoylcarnitine/Myristic acid;
Dodecanoylcarnitine/Bilirubin;
Alanine/Glutamine;
Alanine/1-(1Z-Octadecenyl)-2-oleoyl-sn-glycero-3-phosphocholine;2-Hydroxybutyric acid/Bilirubin;
25-Hydroxyvitamin D3/Ornithine;
2-Hydroxybutyric acid/Cotinine; and
Symmetric dimethylarginine/1-(1Z-Octadecenyl)-2-oleoyl-sn-glycero-3-phosphocholine,
   wherein a ratio of greater than 1 correlates with risk of pre-term pre-eclampsia.

In any embodiment, the ratio is selected from:
Cotinine/2-Hydroxy-(2/3)-methylbutyric acid; and
Bilirubin/2-Hydroxy-(2/3)-methylbutyric acid,
   wherein a ratio of less than 1 correlates with risk of pre-term pre-eclampsia.

In any embodiment, the system comprises a graphical display to display the outputs of the computer processor. In any embodiment, the system comprises a graphical user interface (GUI) to enable a user input data, for example one or more of weight, BMI, racial ethnicity, or the abundance of one or more metabolite biomarkers of the pregnant woman. In any embodiment, the system comprises a device to receive the inputs, a communications module to relay the data to a remote location where the computer processor is located and receive outputs from the computer processor, and a graphical display to display the outputs of the computer processor. The device may be a mobile phone or a computer. In another aspect, the invention provides a computer program, especially a downloadable computer program, for a computation device such as a mobile phone, configured to cause the computation device to receive the inputs, typically via a graphical user interface, communicate the inputs to a remote computer processor via a wired or wireless communication network, and receive outputs from the computer processor via a wired or wireless communication network.

There is also provided a computer program comprising program instructions for causing a computer program to carry out at least one step, and typically all steps, of the method of the invention which may be embodied on a record medium, carrier signal or read-only memory. The embodiments in one aspect of the invention described comprise a system (e.g. a computer apparatus) and/or processes performed in a computer apparatus. However, the invention also extends to computer programs, particularly computer programs stored on or in a carrier adapted to bring the invention into practice. The program may be in the form of source code, object code, or a code intermediate source and object code, such as in partially compiled form or in any other form suitable for use in the implementation of the method according to the invention. The carrier may comprise a storage medium such as ROM, e.g. CD ROM, or magnetic recording medium, e.g. a floppy disk or hard disk. The carrier may be an electrical or optical signal which may be transmitted via an electrical or an optical cable or by radio or other means. The computer program may be downloadable software, for example a downloadable computer program for a mobile phone.

In a further aspect, the system of the invention comprises one or more of the following:
a memory operably connected to the processor and/or the data input module; and
a machine, for example a mass spectrometer module, to determine an abundance of a metabolite from a biological sample obtained from the subject.

In another aspect, the invention provides a pre-eclampsia drug, for example a pre-term pre-eclampsia drug, for example aspirin, for use in a method of preventing pre-term pre-eclampsia in a pregnant woman identified to be at risk of developing pre-term pre-eclampsia according to a method of the invention.

The invention may also be employed to detect risk of pre-term pre-eclampsia at an early stage of pregnancy, for example 8-16 weeks gestation, using the parameters as described above. Early prediction of risk allows early therapeutic intervention optionally combined with increased surveillance and/or lifestyle changes.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human.

Unless otherwise required by context, the use of the terms “AUROC” and “AUC” are used interchangeably herein.

As used herein, the term “at risk of developing pre-term pre-eclampsia” or “risk of pre-term pre-eclampsia” should be understood to mean a risk that is higher than the general population (or sub-population) of pregnant woman. In one embodiment, the term implies an AUROC predictive performance of at least 0.60 or 0.65. In one embodiment, the term implies an AUROC predictive performance of at least 0.70. In one embodiment, the term implies an AUROC predictive performance of at least 0.75. In one embodiment, the term implies an AUROC predictive performance of at least 0.80. In one embodiment, the term implies an AUROC predictive performance of at least 0.85. In one embodiment, the term implies an AUROC predictive performance of at least 0.90.

As used herein, the term “early detection of risk of pre-term pre-eclampsia” means detection of risk prior to the appearance of symptoms of the syndrome, for example during the second trimester of pregnancy (or late first trimester or early third trimester), for example earlier than 20 weeks, or from 8 to 16 weeks, or from 10 and 14 weeks, and ideally about 12 weeks (+/−2 or 3 weeks) gestation. The term “early stage of pregnancy” means prior to the appearance of clinical symptoms of the syndrome, for example during the second trimester of pregnancy, for example from 8 to 16 weeks or from 10 to 14 weeks and ideally about 12 weeks (+/−2, 3, 4 or 5 weeks).

A pregnant woman is diagnosed with pre-eclampsia when the woman has gestational hypertension (systolic BP≥140 mmHg and/or diastolic BP≥90 mmHg (Korotkoff V) on at least 2 occasions 4 h apart after 20 weeks' gestation, but before the onset of labour or postpartum systolic BP≥140 mmHg and/or diastolic BP≥90 mmHg on at least 2 occasions 4 h apart with any of the following new-onset conditions:
proteinuria (≥300 mg/24 h or spot urine protein:creatinine ratio≥30 mg/mmol creatinine, or urine dipstick protein>=++).
Other maternal organ dysfunction, including: Acute kidney Injury (creatinine>=90 micromol/L or >=1 mg/dL); Liver involvement (elevated transaminases, eg, alanine aminotransferase or aspartate aminotransferase>40 IU/L) with or without right upper quadrant or epigastric abdominal pain; Neurological complications (examples include eclampsia, altered mental status, blindness, stroke, clonus, severe headaches, and persistent visual scotomata); Hematological complications (thrombocytopenia-platelet count; <150 000/μL, disseminated intravascular coagulation, hemolysis)
Or Uteroplacental dysfunction (such as fetal growth restriction, abnormal umbilical artery [UA] Doppler wave form analysis, or stillbirth) [13].

As used herein, the term “pre-term pre-eclampsia” should be understood to mean, a pre-eclampsia diagnosis which is made pre-term, e.g. before (<) 37 weeks of gestation and which warrants for the pre-term delivery of the baby, e.g. before (<) 37 weeks of gestation.

As used herein, the term “BMI” or “body mass index” as applied to a pregnant woman should be understood to mean the woman's weight(kg)/(height(m)×(height(m))[Metric system] of the woman's weight(lb)/(height(in)×height(in))×703[Imperial system] taken early in pregnancy, e.g. at 8-24 weeks pregnancy.

As used herein, the term “pre-eclampsia drug” refers to a therapeutic intervention for pregnant women to prevent development of pre-eclampsia typically during the second or third trimester of pregnancy. An Example of therapeutic intervention for preterm pre-eclampsia includes Aspirin. Examples of therapeutic intervention for term pre-eclampsia and/or preeclampsia in the high bmi group include: Low Molecular Weight Heparin; Restriction of weight gain by either caloric intake reduction or life style changes; Interventions to lower the glycemic index, including but not restricted to, insulin, glycemic index lowering probiotics; Citrulline; Antioxidants, including but not limited to, antioxidant vitamins (e.g., ascorbic acid,-tocopherol, -carotene), inorganic antioxidants (e.g., selenium), and a plant-derived polyphenols, antioxidants to mitochondria, including but not limited to, Mito VitE and ergothioneine; statins, including but not limited to, Pravastin. Therapies involving the use of anti-inflammatory or immunosuppressive agents like, but not limited to, tacrolimus, or sulfalazine. In addition, one can easily envision preferred therapeutic combinations like, but not limited to, aspirin and metformin; or metformin and sulfalazine.

As used herein, the term “metformin treatment” refers to treatment with Metformin (GLUCOPHAGE) or a combination therapy comprising Metformin and an addition drug, for example aspirin, thiazolidinediones, DPP-4 inhibitors, sulfonylureas, and meglitinide.

As used herein, the term “white racial origin” refers to a person having origins in any of the original peoples of Europe, the Middle East, or North Africa [https://www.census.gov/topics/population/race/about.html].

The embodiments in the invention described with reference to the drawings comprise a computer apparatus and/or processes performed in a computer apparatus. However, the invention also extends to computer programs, particularly computer programs stored on or in a carrier adapted to bring the invention into practice. The program may be in the form of source code, object code, or a code intermediate source and object code, such as in partially compiled form or in any other form suitable for use in the implementation of the method according to the invention. The carrier may comprise a storage medium such as ROM, e.g. CD ROM, or magnetic recording medium, e.g. a floppy disk or hard disk. The carrier may be an electrical or optical signal which may be transmitted via an electrical or an optical cable or by radio or other means.

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Materials and Methods

### Study Population

This was an observational case-control study drawn from a large prospective screening study on early prediction of complications of pregnancy in women attending for their routine first hospital visit (11-14 weeks of pregnancy) at a hospital in London, UK in the years 2010-2015. Pregnant women received a complete first-trimester assessment including collecting blood samples for 1st trimester biochemical screening and biobanking. Data on pregnancy outcomes were collated for the study participants, the criteria of the American College of Obstetricians and Gynecologists (2019) were used for diagnosis of preeclampsia 20. Written informed consent was obtained from all women; the study was approved by the UK's National Research Ethics Committee; reference number 02-03-033. Within the study all major pregnancy outcomes i.e., preeclampsia, fetal growth restriction, gestational diabetes and spontaneous preterm birth were represented (n=866) as well as uncomplicated pregnancies (n=1635); the latter served as controls in biomarker analyses. Here we report on the nested data for preterm preeclampsia (n=106) vs controls and term preeclampsia (n=267) vs controls.

Descriptive statistics are presented as means (standard deviation, SD), median (Inter Quartile Range, IQR) and frequency of observations (percentages), as appropriate. Comparisons of patient characteristics and pregnancy outcomes between women with preterm preeclampsia, term preeclampsia and controls were performed using Chi-square or Mann Whitney U test as appropriate (Table 1.)

TABLE 1
Baseline characteristics of the study population. Median
(inter-quartile range), count (percentage). ).

| | preterm PE | | term PE | | Controls | | |
|---|---|---|---|---|---|---|---|
| | (N = 106) | | (N = 267) | | (N = 1635) | | |
| Gestational age at | 12.6 | (12.22, 12.98) | 12.6 | (12.3, 13.0) | 12.7 | (12.3, 13.0) | |
| sampling (week) | | | | | | | |
| Maternal age (year) | 30.5 | (27.5, 35.4) | 32.5 | (28.1, 36.2) | 32.1 | (28.4, 35.5) | |
| Race | | | | | | | |
| White | 48 | (45.3%) | 114 | (42.7%) | 1025 | (62.7%) | * |
| Black | 51 | (48.1%) | 130 | (48.7%) | 433 | (26.5%) | |
| South Asian | 4 | (3.8%) | 13 | (4.9%) | 66 | (4.0%) | |
| East Asian | 1 | (0.9%) | 4 | (1.5%) | 52 | (3.2%) | |
| Mixed | 2 | (1.9%) | 6 | (2.2%) | 59 | (3.6%) | |
| Height (cm) | 164 | (160, 167) | 164 | (160, 169) | 165 | (160, 169) | |
| Weight (kg) | 75.2 | (65.8, 87.0) | 75.0 | (64.1, 86.8) | 65.4 | (59.0, 75.7) | * |
| Body mass index | | | | | | | |
| class | | | | | | | |
| <25 kg/m2 | 33 | (31.1%) | 88 | (33.0%) | 944 | (57.7%) | * |
| 25 to <30 kg/m2 | 33 | (31.1%) | 81 | (30.3%) | 419 | (25.6%) | |
| ≥30 kg/m2 | 40 | (37.7%) | 98 | (36.7%) | 272 | (16.6%) | |
| Conception | | | | | | | |
| In vitro fertilization | 7 | (6.6%) | 10 | (3.7%) | 45 | (2.8%) | |
| Ovulation drugs | 1 | (0.9%) | 3 | (1.1%) | 13 | (0.8%) | |
| Smoking | 4 | (3.8%) | 11 | (4.1%) | 91 | (5.6%) | |
| Diabetes mellitus | | | | | | | |
| Type 1 | 0 | (0.0%) | 1 | (0.4%) | 11 | (0.7%) | |
| Type 2 | 4 | (3.8%) | 3 | (1.1%) | 15 | (0.9%) | |
| SLE/APS | 1 | (0.9%) | 1 | (0.4%) | 6 | (0.4%) | |
| Chronic | 15 | (14.2%) | 45 | (16.9%) | 24 | (1.5%) | * |
| hypertension | | | | | | | |
| Family history of | 11 | (10.4%) | 19 | (7.1%) | 58 | (3.5%) | * |
| preeclampsia | | | | | | | |
| Gestational age at | 34.2 | (31.6, 35.7) | 39.2 | (38.2, 40.3) | 39.2 | (38.7, 39.5) | * |
| delivery (week) | | | | | | | |
| Birth weight (g) | 1771 | (1354, 2093) | 3146 | (2855, 3502) | 3295 | (3100, 3515) | * |
| Birth weight centile | 0.48 | (0.03, 10.14) | 33.38 | (11.56, 62.92) | 47.13 | (29.19, 66.87) | * |

*: Chi-square or Mann Whitney U test as appropriate, p < 0.01.

### Biomarker Analyses

First trimester plasma samples of 2501 singleton pregnancies were analyzed with a targeted tandem LC-MS/MS method for metabolite biomarkers (Metabolomic Diagnostics, Ireland) using analytical methodology as previously reported in 19 and patent application EP3749952A2. In brief, biobanked EDTA plasma samples were thawed once on ice and sub-aliquoted in 40 μL aliquots (BRAVO, Agilent Technologies). At that time patient aliquots were also combined in a Pooled Study QC, from which study-wide QC aliquots were created. Aliquots for the 2501 study participants and replicates for 349 randomly selected subjects, were randomized in 38 analytical batches, with each batch featuring 75 clinical samples (inclusive replicates), calibrator samples (n=8), 9 Pooled Study QC samples and 2×2 ordinary QC samples (QC Low, QC high). Metabolite analysis was performed in 38 consecutive days using two LC-MS/MS set-ups (Agilent Technologies) in parallel, as detailed in below sections.

### Selection of Metabolites

Metabolomic Diagnostics operates a dedicated Liquid Chromatography-tandem Mass Spectrometry (LC-MS/MS) based workflow for the targeted analysis of a panel of metabolites which are reported to associate adverse pregnancy outcomes [Based on patent application EP3749952A2]. The target panel is regularly updated with additional metabolites as a result of recent literature reports or with more metabolites becoming assay-able 19. The below table 2 summarises the metabolites selected into the panel for this study, reference to (representative) literature reports wherein these metabolites were associated with adverse pregnancy outcomes are also provided.

TABLE 2
Selection of Metabolites in LC-MS/MS as used in the study;

| | CAS | | |
|---|---|---|---|
| Metabolite | Number | Abbreviation | Compound Class^{a} |
| Acetylcarnitine | 3040-38-8 | CAR(2:0) | Acyl Carnitine |
| Octanoylcarnitine | 25243-95-2 | CAR(8:0) | Acyl Carnitine |
| Decanoylcarnitine | 1492-27-9 | CAR(10:0) | Acyl Carnitine |
| Dodecanoylcarnitine | 25518-54-1 | CAR(12:0) | Acyl Carnitine |
| Palmitoylcarnitine | 6865-14-1 | CAR(16:0) | Acyl Carnitine |
| Stearoylcarnitine | 25597-09-05 | CAR(18:0) | Acyl Carnitine |
| Aminooctanoic acid | 644-90-6 | AmOct | Alpha Amino Acid |
| Asymmetric | 30315- | ADMA | Amino Acid |
| dimethylarginine | 93-6 | | |
| Alanine | 56-41-7 | Ala | Amino Acid |
| Arginine | 74-79-3 | Arg | Amino Acid |
| Citrulline | 372-75-8 | Cit | Amino Acid |
| Ergothioneine | 497-30-3 | Erg | Amino Acid |
| Glutamine | 56-85-9 | Gln | Amino Acid |
| Homo-arginine | 156-86-5 | h-Arg | Amino Acid |
| Isoleucine | 73-32-5 | Ile | Amino Acid |
| Leucine | 61-90-5 | Leu | Amino Acid |
| NG-Monomethyl-L- | 17035- | L-NMMA | Amino Acid |
| arginine | 90-4 | | |
| Lysine | 56-87-1 | Lys | Amino Acid |
| Methionine | 63-68-3 | Met | Amino Acid |
| N6-Acetyl-L-lysine | 692-04-6 | N6Ac-Lys | Amino Acid |
| Ornithine | 3184-13-2 | Orn | Amino Acid |
| Proline | 147-85-3 | Pro | Amino Acid |
| Proline betaine | 471-87-4 | ProB | Amino Acid |
| Symmetric | 30344- | SDMA | Amino Acid |
| dimethylarginine | 004 | | |
| Taurine | 107-35-7 | Tau | Amino Acid |
| Threonine | 72-19-5 | Thr | Amino Acid |
| Bilirubin | 635-65-4 | BiliR | Bilirubin |
| Biliverdin | 114-25-0 | BiliV | Bilirubin |
| Choline | 62-48-1 | CHO | Choline |
| Dilinoleoyl | 15818-46- | DAG(18:2/18:2) | Diacyl |
| glycerol | 9 | | glycerol |
| | 2442-62-8 | | |
| Myristic acid | 544-63-8 | FA(14:0) | Fatty Acid |
| Palmitic acid | 57-10-3 | FA(16:0) | Fatty Acid |
| Palmitoleic acid | 373-49-9 | FA(16:1n7) | Fatty Acid |
| Vaccenic acid | 693-72-1 | FA(18:1n7) | Fatty Acid |
| Oleic acid | 112-80-1 | FA(18:1n9) | Fatty Acid |
| Linoleic acid | 60-33-3 | FA(18:2n6) | Fatty Acid |
| 8,11,14 Eicosatrienoic | 1783-84-2 | FA(20:3n6) | Fatty Acid |
| acid | | | |
| Arachidonic acid | 506-32-1 | FA(20:4n6) | Fatty Acid |
| Eicosapentaenoic acid | 10417-94-4 | FA(20:5n3) | Fatty Acid |
| Docosahexaenoic acid | 6217-54-5 | FA(22:6n3) | Fatty Acid |
| 2-Hydroxy-(2/3)- | 4026-18-0 | 2H(2/3)MBA | Fatty Acid/Hydroxy |
| methylbutyric acid | 3739-30-8 | | Fatty Acid |
| 3-Hydroxy(iso)valeric | 10237-33-1 | 3H(i)VA | Fatty Acid/Hydroxy |
| acid | 625-08-1 | | Fatty Acid |
| 1-(1Z-Octadecenyl)-2- | 799268- | PC(18:1/18:1) | Glycerophosphocholines |
| oleoyl-sn-glycero-3- | 63-6 | | (di-acyl) |
| phosphocholine | | | |
| 1-Palmitoyl-2-hydroxy- | 2507-55-3 | LPC(16:0) | Glycerophosphocholines |
| sn-glycero-3- | | | (mono-acyl) |
| phosphocholine | | | |
| 1-Heptadecanoyl-2- | 50930-23- | LPC(17:0) | Glycerophosphocholines |
| hydroxy-sn-glycero-3- | 9 | | (mono-acyl) |
| phosphocholine | | | |
| 1-Stearoyl-2-hydroxy-sn- | 19420-57- | LPC(18:0) | Glycerophosphocholines |
| glycero-3- | 6 | | (mono-acyl) |
| phosphocholine | | | |
| 1-Oleoyl-2-hydroxy-sn- | 3542-29-8 | LPC(18:1) | Glycerophosphocholines |
| glycero-3- | | | (mono-acyl) |
| phosphocholine | | | |
| 1-(9Z-Octadecenoyl)-sn- | 124262- | PS(18:1) | Glycerophosphoserines |
| glycero-3-phospho-L- | 93-7 | | |
| serine | | | |
| 2-Hydroxybutyric acid | 3347-90-8 | 2HBA | hydroxy Acid (Alpha) |
| Lactic acid | 79-33-4 | LA | Hydroxy Acid (Alpha) |
| 3-Hydroxybutyric acid | 625-72-9 | 3HBA | Hydroxy Acid (Beta) |
| Glycyl-glycine | 556-50-3 | Gly-Gly | Peptide |
| Sphingosine-1- | 26993-30- | S1P | Phosphosphingolipid |
| phosphate | 6 | | |
| Sphinganine-1- | 19794-97- | Sa1P | Phosphosphingolipid |
| phosphate | 9 | | |
| Cotinine | 486-56-6 | COT | pyrrolidinylpyridines |
| Etiocholanolone | 3602-09-3 | Etgluc | Steroidal glycosides |
| glucuronide | | | |
| Urea | 57-13-6 | URE | Urea |
| 25-Hydroxyvitamin D3 | 63183-36- | 25(OH)D3 | Vitamin D |
| | 3 | | |

^{a}Based on Chemical Taxonomy as adopted in ⁴⁵

Reagents, Consumables and Instrumentation

Chemicals and reagents were of High-Performance Liquid Chromatography (HPLC) and Mass Spectrometry (MS) grade or higher and purchased from Fischer Scientific (Ireland), Sigma-Aldrich (Ireland) or Supelco (Merck, Ireland). Metabolite reference materials and stable isotope-labeled standards were purchased from Sigma-Aldrich (Ireland), Avanti Lipids (AL, USA), QMX Laboratories (UK), IsoSciences (PA, USA), Larodan (Sweden), Cambridge Isotopes Laboratories (UK), TRC Chemicals (ON, Canada), LGC Standards (UK). Cryovials (Wilmut) were sources from Deltalab (Spain), all other plastics (tips, plates, mats) from Agilent Technologies (Ireland) or Sigma-Aldrich (Ireland).

For sample preparation, a liquid handling robot (BRAVO), equipped with a 96 LT disposable Tip Head, an orbital shaker station and a Peltier thermal station, was used (Agilent Technologies, CA, USA). Targeted metabolite LC-MS/MS analysis was performed using Multiple Reaction Monitoring (MRM) on two equivalent platforms, each consisting of a 1260 (II) Infinity LC system (Agilent Technologies, Germany) coupled to a Triple Quadrupole 6460 (A/C) mass spectrometer (QqQ-MS) equipped with a Jet Stream Electrospray Ionisation (ESI) source (Agilent Technologies, CA, USA (A) or Germany (C)).

### Preparations

The mixture of Stable Isotope-Labeled Internal Standards (SIL-IS) was prepared in accordance with ¹⁹. Calibrators (n=8) containing all target metabolites were prepared in in 5% Bovine Serum Albumin in Phosphate-buffered Saline (Sigma-Aldrich, Ireland); with calibration ranges estimated from a test batch. Quality control samples (QC-High & QC-Low) were prepared with QC-High at a level of about 70% of highest calibrator and QC-Low at a level of about 15%.

### Metabolite Analyses

Metabolite Analysis was performed using methodology as reported in Kenny et al. ¹⁹. Upon semi-automated sample preparation (BRAVO), i.e., addition of mixture of SIL-IS, incubation, protein precipitation with organic solvent followed by centrifugation, the resulting metabolite extracts were taken off (liquid handling robot) and equally split over 2 classic 96 well plates. These twin plates were dried under vacuum (speedvac, Labconco, Ireland) and then separately reconstituted before parallel analysis with a Reversed Phase Liquid Chromatography-tandem Mass Spectrometry method (RPLC-MS/MS) and Hydrophilic Interaction Liquid Chromatography-tandem Mass Spectrometry method respectively (HILIC-MS/MS). The RPLC method remained unchanged ¹⁹. To increase method robustness and number of metabolites amenable to analysis the HILIC chromatography was further optimised. This involved the use of two HILIC mounted in series and repeat injecting the sample. Columns used where Agilent Poroshell Hilic-Z 15 cm×2.1 mm, 27 micron particles (Agilent Technologies, Ireland) and Phenomenex Biozen Glycan 10 cm×2.1 mm×26 micron particles (Phenomenex, Ireland); mobile phases A: 50 mM Ammonium formate; B: Acetonitrile; 10 min gradient program, injections at time 0 and at 6 minutes. Both RPLC- and HILIC-eluates were directly fed to the ESI-QqQ-MS. ESI polarity switching was applied in function of metabolite ionisation characteristics.

LC-MRM assay parameters for the metabolites and SIL-IS of the respective RPLC- and HILIC-MS/MS are summarised below (Retention time (Rt) in minutes; ESI polarity, positive (pos) or negative (neg); MRM transitions for both quantifier (Quant) and qualifier (Qual) transitions; for the SIL-IS the type of isotopic label(s) and number of labels are also provided.

Data pre-Processing and Quality Assurance-Summary Quality Control

Following mass spectrometric analysis, the mass spectrometric signals were quantified using a pre-defined quantification method (Masshunter Quant Software, Agilent Technologies). Data were then reviewed by two independent analysts; all manual curation was recorded for data integrity purposes. Laboratory personnel were blinded to sample status (pregnancy outcome) at all stages of the study. Structured review of all mass spectrometry was performed to confirm the quantification metric to use. For each metabolite (Met) assayed and its respective assigned SIL-IS, it was verified that the assigned SIL-IS was fit-for-purpose in terms of correcting for technical variability, the chosen quantifier and qualifier transitions supported (relative) quantification and whether there was a need for additional inter-batch normalisation. When required, alternative SIL-IS were evaluated and implemented as appropriate. Metabolites with too low signal counts in metabolite quantifier transitions were removed. For Proline (Pro) loss of correlation between quantifier and qualifier data was observed at high blood levels indicative for detector overload in the quantifier transition; role of quantifier and qualifier transitions were exchanged. Pairwise dependencies between metabolite quantification metrics and recorded experimental variables were computed using, as appropriate, Spearman's rank correlation, MWU test, or Kruskal-Wallis test; only minor inter-day batch effects in some of the quantifications were found. The decision point to consider application of inter-batch normalisation was arbitrarily set at an improvement in % CV of >=5% as estimated from the 349 replicate analyses with replicates randomly distributed over the 38 batches. For these, the relative concentrations were scaled per batch using the median concentration of the 9 Pooled Study QC samples for the given batch over the overall median concentration ⁴⁶. Data missingness and imprecision criteria were applied, except for cotinine a reporter metabolite for smoking status, with data missingness for a given metabolite quantification<20% across all clinical samples and % CV≤20% as calculated for the 349 pairs of replicate samples. Finally, metabolite quantifications from LC-MS/MS assay with poorly understood specificity were also purged. Below table summarises the mass spectrometry data review and quality assurance applied. The % CV reported corresponds to the estimated % CV from the 349 replicate analyses upon implementation of all technical review decisions, i.e., SIL-IS selection, quantification metric selection and inter-batch normalisation.

### Data Pre-Processing and Quality Assurance

Following quality control, quantification data for 50 metabolites across 15 chemical classes was available for prediction analysis with amino acids (n=17), fatty acids (n=8), acyl carnitines (n=6), and mono-acyl glycerophosphocholines (n=4), accounting for 60% of the metabolites. Two pairs of structural isomers were not analytically resolved and were analysed as single analytes, i.e., 3-hydroxy (iso) valeric acid and 2-hydroxy-(2/3)-methylbutyric acid. The same applied to the three structural- and stereoisomers of dilinoleoyl glycerol.

### Composite Biomarkers

An additional set of composite biomarkers was created by taking the pairwise ratios of all metabolites analysed.

### Confounding Associations

Biomarker data, i.e., single metabolites and metabolite ratios, were normalized using multiple of medians (MoM) on storage age of the sample, gestation age at sampling, maternal age, weight and BMI, smoking and racial origin 47. Log-normality was confirmed in control subjects using Shapiro-Wilk test and Quantile-Quantile plots. Then parameters for MOM normalization of a given biomarker were selected using multiway analysis of variance (ANOVA) on the control pregnancies (p<0.001). The normalization coefficients were computed and applied to all study participants. Cotinine was excluded from normalization. For biomarkers without confounding associations, read-outs were transformed into simple medians on the controls.

### Prediction Analysis

Prediction analyses were performed across the following subject strata: all subjects, normal weight(BMI<25), overweight(25≤BMI<30), obese (BMI≥30), Afro-Caribbean, White. No sufficient preeclampsia cases were available to analyze prediction in other race strata. Within a stratum the discriminative performances of biomarkers were firstly estimated with Mann-Whitney U test by looking at the fold change of normalized biomarker concentrations, i.e., single metabolites or metabolite ratios, in EDTA plasma samples of pregnant women who went on to develop preeclampsia later in pregnancy vs. pregnant women who went on to have an uncomplicated delivery (reference). The discriminative performance of the biomarkers, i.e. metabolites and metabolite ratios, was quantified using the area under the receiver operating curve (AUROC) 48. An AUROC of 0.5 or lower indicates an absence of predictive power for the outcome. The 95% Confidence Interval for the Area Under the Curve was calculated. A biomarker is considered a predictor if its AUROC was significantly higher than 0.5 (p<0.05). The following significance levels were considered: * p<0.05, ** p<0.01, *** p<0.001, **** p<0.0001 Software: Statistical analyses were performed in R49

### Results

Preterm preeclampsia prediction using metabolites in pregnancy population stratum: “All”.

TABLE 3: Single Metabolite preterm preeclampsia predictors in pregnancy population stratum: “All”

In TABLE 3, the single metabolites are reported which, upon correction of any confounding associations, have preterm preeclampsia prediction performance as evaluated by the statistic AUROC. From TABLE 3 it is apparent that single metabolites that all but one metabolite (CAR (10:0)) needed correction for confounding factors, and that single metabolites have limited prediction performance when it comes to predicting preterm preeclampsia in pregnant women. From TABLE 3, it is clear that, upon correction for confounders, increased blood levels of 25(OH) D, 2H(2/3) MBA, 2HBA, Ala, CAR (10:0), 3H (i) VA, Erg, CAR (8:0) and Thr, and decreased blood levels of PC(18:1/18:1), BiliR and Gln associated with increased risk of developing preterm preeclampsia later in pregnancy.

TABLE 3
Prediction of Preterm preeclampsia in all pregnant women with single
metabolites; AUROC and Median Fold Changes Cases/Controls

| | Correction for | | Median Fold Change |
|---|---|---|---|
| | confounding | AUROC | Cases over Controls |
| Metbolite | (MoM) | (95% CI) | (95% CI) |
| 25(OH)D3 | Yes | 0.60 | 1.16 |
| | | (0.54-0.66) *** | (1.07-1.26) *** |
| 2H(2/3)MBA | Yes | 0.60 | 1.17 |
| | | (0.55-0.66) *** | (1.06-1.22) *** |
| 2HBA | Yes | 0.60 | 1.12 |
| | | (0.54-0.65) *** | (1.06-1.26) *** |
| CAR(12:0) | Yes | 0.59 | 1.22 |
| | | (0.53-0.64) ** | (1.08-1.43) ** |
| Ala | Yes | 0.59 | 1.07 |
| | | (0.53-0.65) ** | (1.02-1.10) ** |
| PC(18:1/18:1) | Yes | 0.59 | 0.93 |
| | | (0.53-0.64) ** | (0.89-0.98) ** |
| BiliR | Yes | 0.57 | 0.89 |
| | | (0.51-0.62) * | (0.81-0.99) * |
| CAR(10:0) | No | 0.57 | 1.18 |
| | | (0.51-0.62) * | (1.02-1.43) * |
| 3H(i)VA | Yes | 0.57 | 1.12 |
| | | (0.51-0.62) * | (1.02-1.24) * |
| Erg | Yes | 0.57 | 1.16 |
| | | (0.51-0.62) * | (1.02-1.33) * |
| CAR(8:0) | Yes | 0.56 | 1.18 |
| | | (0.50-0.61) * | (1.01-1.37) * |
| Thr | Yes | 0.56 | 1.05 |
| | | (0.51-0.62) * | (1.00-1.11) * |
| Gln | Yes | 0.56 | 0.95 |
| | | (0.50-0.62) * | (0.93-1.00) * |

TABLE 4: Metabolite ratios as preterm preeclampsia predictors in pregnancy population stratum: “All”

In keeping with the insight that metabolite ratios may have more discriminative power than single metabolites, metabolite ratios were assessed as preterm preeclampsia predictors as well. Given the aim to discover more effective and robust parameters for assessing preeclampsia risk, we required the metabolite ratios to deliver better preterm preeclampsia risk prediction, as estimated by AUROC, than the best single markers in TABLE 3 therefore only metabolite ratios with AUROC>0.6 were considered.

In TABLE 4, the metabolite ratios are reported which, upon correction of any confounding associations, have preterm preeclampsia prediction performance higher than AUROC>0.6. Confirming the validity of our insights, a multitude of metabolite ratios were found as significant predictors, typically with higher levels of significance and larger fold changes, compared to the single metabolites. Again, it was also found that most of the metabolite ratios required correction for confounding, with exception of the ratio Ala/Gln. It is evident that when it concerns ratios of metabolite blood levels, it is not important which metabolite level is the numerator and which metabolite level is the denominator. From Table 4, it is apparent that the following ratios of metabolite blood levels associate with preterm preeclampsia risk:ratios constituting PC(18:1/18:1) and any of 25(OH)D3, 2HBA, Ala, SDMA, 2H(2/3) MBA; additionally ratios constituting 2H(2/3) MBA and any of BiliR, COT, Gln, Orn; additionally ratios constituting Gln and 25(OH)D3 or Ala; additionally ratios constituting BiliR and 2HBA or CAR (12:0), as well as the additional ratios of 25(OH)D3 and Orn: 2HBA or COT; CAR (12:0) and FA (14:0).

TABLE 4
Prediction of Preterm preeclampsia in all pregnant women with
metabolite ratios; AUROC and Median Fold Changes Cases/Controls

| | Correction | | |
|---|---|---|---|
| | for | | Median Fold Change |
| | confounding | AUROC | Cases over Controls |
| Metabolite Ratio | (MoM) | (95% CI) | (95% CI) |
| 25(OH)D3/ | Yes | 0.65 | 1.32 |
| PC(18:1/18:1) | | (0.59-0.71) **** | (1.17-1.42) **** |
| 2H(2/3)MBA/Orn | Yes | 0.63 | 1.24 |
| | | (0.58-0.68) **** | (1.13-1.35) **** |
| 2H(2/3)MBA/ | Yes | 0.63 | 1.24 |
| PC(18:1/18:1) | | (0.57-0.68) **** | (1.12-1.34) **** |
| 25(OH)D3/Gln | Yes | 0.62 | 1.28 |
| | | (0.56-0.68) *** | (1.10-1.33) **** |
| 2H(2/3)MBA/Gln | Yes | 0.62 | 1.20 |
| | | (0.56-0.67) **** | (1.09-1.29) **** |
| 2HBA/PC(18:1/18:1) | Yes | 0.62 | 1.20 |
| | | (0.56-0.67) **** | (1.11-1.35) **** |
| CAR(12:0)/FA(14:0) | Yes | 0.62 | 1.28 |
| | | (0.56-0.67) **** | (1.13-1.40) **** |
| CAR(12:0)/BiliR | Yes | 0.62 | 1.38 |
| | | (0.56-0.67) **** | (1.17-1.58) **** |
| Ala/Gln | No | 0.62 | 1.09 |
| | | (0.56-0.67) **** | (1.05-1.15) **** |
| Ala/PC(18:1/18:1) | Yes | 0.62 | 1.15 |
| | | (0.57-0.68) **** | (1.08-1.22) **** |
| 2HBA/BiliR | Yes | 0.61 | 1.26 |
| | | (0.55-0.66) *** | (1.12-1.46) *** |
| BiliR/2H-(2/3)MBA | Yes | 0.61 | 0.79 |
| | | (0.55-0.67) *** | (0.69-0.89) *** |
| 25(OH)D3/Orn | Yes | 0.61 | 1.23 |
| | | (0.55-0.67) *** | (1.10-1.36) *** |
| 2HBA/COT | Yes | 0.61 | 1.36 |
| | | (0.56-0.67) **** | (1.15-1.52) **** |
| COT/2H(2/3)MBA | Yes | 0.61 | 0.80 |
| | | (0.55-0.66) *** | (0.70-0.89) *** |
| SDMA/PC(18:1/18:1) | Yes | 0.61 | 1.13 |
| | | (0.55-0.66) *** | (1.06-1.19) *** |

### Preterm Preeclampsia Prediction Using Metabolites in Pregnancy Population Strata: “BMI<25” and “BMI≥30”

In keeping with the inventors understanding that the epidemiologically observed differences in preeclampsia rates in pregnant women across different BMI classes may reflect the existence of different underlying dominant maternal risk profile in pregnant women with different BMI, the existence of blood biomarkers which associate particularly strong with preeclampsia risk in women with a certain BMI and not women in women with another different BMI can be imagined. Therefore, the inventors looked for metabolites and metabolites ratios which associate strongly with preterm preeclampsia in two mutually exclusive strata of pregnant women, i.e., pregnant women with BMI<25 early in pregnancy, or in women with BMI≥30 early in pregnancy, whereby the first stratum (BMI<25) aggregates women classified as “underweight” and “normal weight” and the second stratum (BMI≥30) aggregates women classified as “obese”, in accordance with the World Health Organisation BMI classification ⁵⁰. It is noted that in Asian populations, different BMI cut-off are used to classify people in weight classes⁵¹.

### TABLE 5: Single Metabolites as Preterm Preeclampsia Predictors in Pregnancy Population Stratum: “BMI<25”

To elicit the existence of metabolite biomarkers which are specifically associating with preterm preeclampsia risk in the pregnancy population stratum “BMI<25”, we required for any given metabolite that the preterm preeclampsia risk prediction, as estimated by AUROC, was at least 0.07 AUROC units (7%) higher in the “BMI<25” stratum compared to the AUROC for the same metabolite in the “BMI≥30” stratum. Given the aim to discover more effective and robust parameters for assessing preeclampsia risk, we required the metabolite ratios to deliver better preterm preeclampsia risk prediction in the “BMI<25” stratum, as estimated by AUROC, than the best single markers in the unstratified pregnancy population therefore only metabolites with AUROC>0.6 were considered.

In TABLE 5, the single metabolite is reported which, upon correction of any confounding associations, has preterm preeclampsia prediction performance, as evaluated by the statistic AUROC, which is specific to the “BMI<25” stratum. It is found that Orn blood levels only associate with preterm preeclampsia risk in women of the BMI<25 stratum, whereby decreased Orn blood levels increase the risk of preterm preeclampsia later in pregnancy. It is noted that Orn is not identifiable as a biomarker for preterm preeclampsia risk when assessed in the unstratified pregnancy population. Its exceptional association with preterm preeclampsia risk is confined to pregnant in the aggregated BMI classification “underweight” and “normal weight” and would not be exposed without application of the prior stratification of the pregnancy population in BMI groups.

TABLE 5
Prediction of Preterm preeclampsia in BMI < 25 pregnant women with metabolites; AUROC and Median Fold Changes Cases / Controls

| | Prior correction | | | | | Fold Change | | | |
|---|---|---|---|---|---|---|---|---|---|
| | for confounding | AUROC (95% CI) | | | | Cases over Controls (95% CI) | | | |
| Metabolite | (MoM) | All | BMI < 25 | 25 =< BMI < 30 | BMI >= 30 | All | BMI < 25 | 25 =< BMI < 30 | BMI >= 30 |
| Orn | Yes | 0.52 | 0.69 | 0.54 | 0.51 | 0.98 | 0.87 | 1.03 | 1.03 |
| | | (0.47- | (0.60- | (0.44-0.64) | (0.41-0.60) | (0.92- | (0.76- | (0.94-1.15) | (0.92-1.11) |
| | | 0.58) | 0.77) **** | | | 1.03) | 0.92) *** | | |

### TABLE 6: Metabolite Ratios as Preterm Preeclampsia Predictors in Pregnancy Population Stratum: “BMI<25”

Metabolite ratios were assessed as preterm preeclampsia predictors specific to pregnant women in the BMI<25 stratum as well. Again, we required for any given metabolite ratio that the preterm preeclampsia risk prediction, as estimated by AUROC, was at least 0.07 AUROC units (7%) higher in the “BMI<25” stratum compared to the AUROC for the same metabolite ratio in the “BMI≥30” stratum. Given the aim to discover more effective and robust parameters for assessing preeclampsia risk, we required the metabolite ratios to deliver better preterm preeclampsia risk prediction in the “BMI<25” stratum, as estimated by AUROC, than the best single marker Orn in TABLE 5 when Orn is part of the ratio (GROUP A; AUROC>0.69) OR when the metabolite ratio delivered an AUROC>0.6 together with a significance level of ** or better (GROUP B).

In TABLE 6, the metabolite ratios are reported which, upon correction of any confounding associations, have preterm preeclampsia prediction performance, as evaluated by the statistic AUROC, which is specific to the “BMI<25” stratum. From Table 6, it is apparent that the following ratios of metabolite blood levels (Group A) associate with preterm preeclampsia risk and improve the specific preterm preeclampsia prediction of the single maker Orn (TABLE 5), i.e., ratios constituting Orn and any of Thr, ADMA, LPC(18:1), LPC(16:1), Ala and Gln. In addition, the following metabolite ratios associate significantly with preterm preeclampsia risk in the BMI<25 stratum, i.e., ratios constituting Cit and any of LPC(18:1), LPC(16:0), 25(OH)D3 or h-Arg, additionally ratios constituting LPC(18:1) and any of PC(18:1/18:1) or PS (18:1).

TABLE 6
Prediction of Preterm preeclampsia in BMI < 25 pregnant women with metabolite ratios; AUROC and Median Fold Changes Cases / Controls

| | | | | | | | Fold Change | | | |
|---|---|---|---|---|---|---|---|---|---|---|
| | Correction for | | AUROC (95% CI) | | | | Cases over Controls (95% CI) | | | |
| | confounding | | | | 25 =< | | | | 25 =< | |
| Metabolite Ratio | (MoM) | Group | All | BMI < 25 | BMI < 30 | BMI >= 30 | All | BMI < 25 | BMI < 30 | BMI >= 30 |
| Thr / Orn | Yes | A | 0.60 | 0.72 | 0.52 | 0.59 | 1.11 | 1.28 | 0.97 | 1.09 |
| | | | (0.55- | (0.63- | (0.43- | (0.50- | (1.05- | (1.14- | (0.88- | (0.99- |
| | | | 0.66) *** | 0.81) **** | 0.62) | 0.68) | 1.17) *** | 1.39) **** | 1.09) | 1.19) |
| ADMA / Orn | Yes | A | 0.57 | 0.71 | 0.50 | 0.55 | 1.07 | 1.19 | 0.99 | 1.01 |
| | | | (0.52- | (0.62- | (0.41- | (0.46- | (1.01- | (1.10- | (0.91- | (0.96- |
| | | | 0.63) ** | 0.79) **** | 0.60) | 0.63) | 1.12) * | 1.29) **** | 1.09) | 1.13) |
| LPC(18:1) / | Yes | A | 0.56 | 0.71 | 0.50 | 0.51 | 1.13 | 1.29 | 1.07 | 0.96 |
| Orn | | | (0.49- | (0.62- | (0.38- | (0.42- | (1.00- | (1.17- | (0.86- | (0.85- |
| | | | 0.62) | 0.81) **** | 0.61) | 0.61) | 1.17) | 1.52) **** | 1.16) | 1.12) |
| LPC(16:0) / | Yes | A | 0.55 | 0.71 | 0.53 | 0.49 | 1.05 | 1.29 | 0.97 | 0.99 |
| Orn | | | (0.49- | (0.62- | (0.42- | (0.39- | (0.99- | (1.13- | (0.86- | (0.92- |
| | | | 0.61) | 0.80) **** | 0.64) | 0.58) | 1.12) | 1.36) **** | 1.09) | 1.13) |
| Ala / Orn | Yes | A | 0.58 | 0.70 | 0.51 | 0.57 | 1.09 | 1.29 | 1.02 | 1.07 |
| | | | (0.52- | (0.60- | (0.40- | (0.47- | (1.03- | (1.11- | (0.91- | (0.98- |
| | | | 0.64) ** | 0.79) **** | 0.62) | 0.67) | 1.15) ** | 1.37) *** | 1.13) | 1.18) |
| Gln / Orn | Yes | A | 0.52 | 0.70 | 0.56 | 0.54 | 0.97 | 0.82 | 1.05 | 1.02 |
| | | | (0.46- | (0.61- | (0.46- | (0.44- | (0.92- | (0.76- | (0.96- | (0.94- |
| | | | 0.58) | 0.79) **** | 0.66) | 0.64) | 1.04) | 0.91) *** | 1.19) | 1.15) |
| Cit / LPC(18:1) | Yes | B | 0.54 | 0.67 | 0.53 | 0.49 | 0.90 | 0.86 | 1.04 | 0.96 |
| | | | (0.48- | (0.58- | (0.42- | (0.39- | (0.87- | (0.72- | (0.89- | (0.88- |
| | | | 0.60) | 0.76) *** | 0.64) | 0.59) | 1.02) | 0.91) *** | 1.21) | 1.17) |
| 25(OH)D3 / Cit | Yes | B | 0.59 | 0.64 | 0.55 | 0.56 | 1.16 | 1.28 | 1.04 | 1.12 |
| | | | (0.53- | (0.54- | (0.44- | (0.45- | (1.06- | (1.07- | (0.91- | (0.92- |
| | | | 0.65) ** | 0.73) ** | 0.65) | 0.67) | 1.30) ** | 1.50) ** | 1.31) | 1.36) |
| LPC(18:1) / | Yes | B | 0.58 | 0.63 | 0.56 | 0.56 | 1.10 | 1.08 | 1.12 | 1.15 |
| PC(18:1/18:1) | | | (0.52- | (0.54- | (0.45- | (0.46- | (1.03- | (1.04- | (0.95- | (0.95- |
| | | | 0.64) | 0.71) ** | 0.67) | 0.67) | 1.21) ** | 1.33) * | 1.26) | 1.27) |
| PS(18:1) / | Yes | B | 0.53 | 0.63 | 0.50 | 0.54 | 0.99 | 0.92 | 0.98 | 1.08 |
| LPC(18:1) | | | (0.47- | (0.54- | (0.38- | (0.45- | (0.94- | (0.85- | (0.91- | (0.95- |
| | | | 0.59) | 0.72) ** | 0.62) | 0.63) | 1.02) | 0.98) ** | 1.09) | 1.13) |
| Cit / LPC(16:0) | Yes | B | 0.52 | 0.63 | 0.57 | 0.49 | 0.96 | 0.90 | 1.12 | 1.01 |
| | | | (0.46- | (0.54- | (0.45- | (0.39- | (0.92- | (0.81- | (0.95- | (0.89- |
| | | | 0.58) | 0.72) ** | 0.68) | 0.59) | 1.04) | 0.97) * | 1.22) | 1.10) |
| Cit / h-Arg | Yes | B | 0.51 | 0.63 | 0.60 | 0.51 | 0.98 | 0.79 | 1.32 | 0.98 |
| | | | (0.45- | (0.53- | (0.48- | (0.41- | (0.89- | (0.67- | (0.99- | (0.83- |
| | | | 0.57) | 0.73) ** | 0.71) | 0.61) | 1.09) | 0.95) * | 1.44) | 1.17) |

### TABLE 7: Single Metabolites as Preterm Preeclampsia Predictors in Pregnancy Population Stratum: “BMI≥30”

To elicit the existence of metabolite biomarkers which are specifically associating with preterm preeclampsia risk in the pregnancy population stratum “BMI>30”, we required for any given metabolite that the preterm preeclampsia risk prediction, as estimated by AUROC, was at least 0.07 AUROC units (7%) higher in the “BMI>30” stratum compared to the AUROC for the same metabolite in the “BMI<25” stratum. Given the aim to discover more effective and robust parameters for assessing preeclampsia risk, we required the metabolite ratios to deliver better preterm preeclampsia risk prediction in the “BMI>30” stratum, as estimated by AUROC, than the best single markers in the unstratified pregnancy population therefore only metabolite ratios with AUROC>0.6 were considered.

In TABLE 7, the single metabolites are reported which, upon correction of any confounding associations, have preterm preeclampsia prediction performance, as evaluated by the statistic AUROC, which are specific to the “BMI>30” stratum. It is found that 2H(2/3) MBA, CAR (10:0), CAR (12:0), Ala and SDMA blood levels only associate with preterm preeclampsia risk in women of the BMI>30 stratum, whereby increased blood levels of these metabolites increase the risk of preterm preeclampsia later in pregnancy.

TABLE 7
Prediction of Preterm preeclampsia in BMI ≥ 30 pregnant women with metabolites; AUROC and Median Fold Changes Cases / Controls

| | Correction for | | | | | Fold Change | | | |
|---|---|---|---|---|---|---|---|---|---|
| | confounding | AUROC (95% CI) | | | | Cases over Controls (95% CI) | | | |
| Metabolite | (MoM) | All | BMI < 25 | 25 =< BMI < 30 | BMI >= 30 | All | BMI < 25 | 25 =< BMI < 30 | BMI >= 30 |
| 2H(2/3)MBA | Yes | 0.60 | 0.58 | 0.53 | 0.67 | 1.17 | 1.22 | 1.05 | 1.22 |
| | | (0.55- | (0.48- | (0.44-0.62) | (0.58- | (1.06- | (0.98- | (0.92-1.17) | (1.12- |
| | | 0.66) *** | 0.69) | | 0.76) *** | 1.22) *** | 1.27) | | 1.46) *** |
| CAR(12:0) | Yes | 0.59 | 0.56 | 0.49 | 0.64 | 1.22 | 1.30 | 0.99 | 1.39 |
| | | (0.53- | (0.46- | (0.38-0.59) | (0.55- | (1.08- | (0.90- | (0.80-1.32) | (1.11- |
| | | 0.64) ** | 0.66) | | 0.73) ** | 1.43) ** | 1.52) | | 1.73) ** |
| Ala | Yes | 0.59 | 0.52 | 0.60 | 0.63 | 1.07 | 1.04 | 1.08 | 1.11 |
| | | (0.53- | (0.42- | (0.51-0.69) * | (0.53- | (1.02- | (0.95- | (1.00-1.12) | (1.02- |
| | | 0.65) ** | 0.63) | | 0.72) ** | 1.10) ** | 1.09) | | 1.15) * |
| CAR(10:0) | No | 0.57 | 0.54 | 0.50 | 0.61 | 1.18 | 1.14 | 0.85 | 1.36 |
| | | (0.51- | (0.44- | (0.40-0.61) | (0.52- | (1.02- | (0.83- | (0.74-1.33) | (1.03- |
| | | 0.62) * | 0.64) | | 0.69) * | 1.43) * | 1.54) | | 1.77) * |
| SDMA | Yes | 0.52 | 0.54 | 0.49 | 0.61 | 1.00 | 0.97 | 1.01 | 1.04 |
| | | (0.46- | (0.44- | (0.38-0.60) | (0.52- | (0.98- | (0.93- | (0.93-1.06) | (1.01- |
| | | 0.58) | 0.64) | | 0.71) * | 1.05) | 1.03) | | 1.13) * |

### TABLE 8: Metabolite Ratios as Preterm Preeclampsia Predictors in Pregnancy Population Stratum: “BMI≥30”

Metabolite ratios were assessed as preterm preeclampsia predictors specific to pregnant women in the BMI≥30 stratum as well. Again, we required for any given metabolite ratio that the preterm preeclampsia risk prediction, as estimated by AUROC, was at least 0.07 AUROC units (7%) higher in the “BMI≥30” stratum compared to the AUROC for the same metabolite ratio in the “BMI<25” stratum. Given the aim to discover more effective and robust parameters for assessing preeclampsia risk, we required the metabolite ratios to deliver better preterm preeclampsia risk prediction in the “BMI≥30” stratum, as estimated by AUROC, than the respective single markers reported in TABLE 7. Hereby any metabolite ratio constituting any of the single markers reported in TABLE 7 should have an improved preterm preeclampsia prediction, as estimated by AUROC. In addition, the metabolite ratio should have an AUROC>0.6 in the BMI≥30 stratum and a significance level of ** or better. Accordingly, the following groups of metabolite ratios were formulated:
GROUP A: AUC>0.67
GROUP B: AUC>0.64, excluding any metabolite ratios constituting 2H(2/3) MBA
GROUP C: AUC>0.63, excluding any metabolite ratios constituting 2H(2/3) MBA or CAR (12:0)·
GROUP D: AUC>0.61, excluding any metabolite ratios constituting 2H(2/3) MBA, CAR (12:0) or Ala

In TABLE 8, the metabolite ratios are reported which, upon correction of any confounding associations, have preterm preeclampsia prediction performance, as evaluated by the statistic AUROC, which is specific to the “BMI≥30” stratum. From Table 8, it is apparent that the ratios of metabolite blood levels constituting 2H(2/3) MBA and PC(18:1/18:1) or Gln further improve the specific preterm preeclampsia prediction of the single maker 2H(2/3) MBA (TABLE7) in the BMI≥30 stratum. Similarly, ratios constituting CAR (12:0) and FA (14:0), FA (16:0) or PC(18:1/18:1) improve the prediction of the single marker CAR (12:0) (TABLE 7).

Likewise, the metabolite ratio constituting Ala and GIn improves the prediction of the single marker Ala (TABLE 7); the metabolite ratios constituting SDMA and PC(18:1/18:1) and Gln improve the prediction of the single marker SDMA (TABLE 7) and the metabolite ratios constituting CAR (10:0) and PC(18:1/18:1) and FA (14:0) 5 improve the prediction of the single marker CAR (10:0) (TABLE 7). Interestingly, all other metabolite ratios with associate strongly with preterm preeclampsia risk in the “BMI≥30” stratum either constitute PC(18:1/18:1) or Gln, whereby additional metabolite ratios constituting PC(18:1/18:1) and any of 2HBA, Met, CAR (18:0), Arg, ADMA, Pro, CAR (16:0), or L-NMMA and metabolite ratios constituting Gln and any of Met, 2HBA or Arg were identified.

TABLE 8
Prediction of Preterm preeclampsia in BMI ≥ 30 pregnant women with metabolite ratios; AUROC and Median Fold Changes Cases / Controls

| | | | | | | | Fold Change | | | |
|---|---|---|---|---|---|---|---|---|---|---|
| | Correction for | | AUROC (95% CI) | | | | Cases over Controls (95% CI) | | | |
| | confounding | | | | 25 =< | | | | 25 =< | |
| Metabolite Ratio | (MoM) | Group | All | BMI < 25 | BMI < 30 | BMI >= 30 | All | BMI < 25 | BMI < 30 | BMI >= 30 |
| 2H(2/3)MBA / | Yes | A | 0.63 | 0.62 | 0.57 | 0.70 | 1.24 | 1.24 | 1.04 | 1.45 |
| PC(18:1/18:1) | | | (0.57- | (0.52- | (0.47- | (0.62- | (1.12- | (1.03- | (0.94- | (1.22- |
| | | | 0.68) **** | 0.72) * | 0.66) | 0.79)**** | 1.34) **** | 1.41)* | 1.29) | 1.71) **** |
| 2H(2/3)MBA / | Yes | A | 0.62 | 0.59 | 0.55 | 0.69 | 1.20 | 1.25 | 1.05 | 1.38 |
| Gln | | | (0.56- | (0.48- | (0.46- | (0.61- | (1.09- | (0.98- | (0.93- | (1.18- |
| | | | 0.67) **** | 0.70) | 0.64) | 0.78)**** | 1.29) **** | 1.34) | 1.23) | 1.60) **** |
| 2HBA / | No | A | 0.62 | 0.60 | 0.58 | 0.68 | 1.20 | 1.19 | 1.17 | 1.23 |
| PC(18:1/18:1) | | | (0.56- | (0.49- | (0.48- | (0.60- | (1.11- | (1.00- | (0.97- | (1.15- |
| | | | 0.67) **** | 0.71) | 0.69) | 0.76)**** | 1.35) **** | 1.44) | 1.39) | 1.54) *** |
| CAR(12:0) / | No | A | 0.62 | 0.56 | 0.57 | 0.68 | 1.28 | 1.07 | 1.16 | 1.49 |
| FA(14:0) | | | (0.56- | (0.47- | (0.46- | (0.59- | (1.13- | (0.93- | (0.94- | (1.19- |
| | | | 0.67) **** | 0.65) | 0.68) | 0.76)**** | 1.40) **** | 1.36) | 1.37) | 1.71) *** |
| SDMA / | Yes | B | 0.61 | 0.60 | 0.57 | 0.67 | 1.13 | 1.11 | 1.08 | 1.22 |
| PC(18:1/18:1) | | | (0.55- | (0.52- | (0.46- | (0.57- | (1.06- | (1.00- | (0.96- | (1.10- |
| | | | 0.66) *** | 0.69)* | 0.68) | 0.77)*** | 1.19) *** | 1.19) * | 1.21) | 1.37) *** |
| Met / Gln | Yes | B | 0.58 | 0.48 | 0.57 | 0.67 | 1.12 | 1.02 | 1.06 | 1.22 |
| | | | (0.53- | (0.37- | (0.47- | (0.58- | (1.03- | (0.88- | (0.97- | (1.07- |
| | | | 0.64) ** | 0.59) | 0.67) | 0.75)*** | 1.14) ** | 1.09) | 1.17) | 1.27) *** |
| CAR(12:0) / | Yes | B | 0.59 | 0.58 | 0.48 | 0.66 | 1.28 | 1.27 | 0.84 | 1.36 |
| PC(18:1/18:1) | | | (0.53- | (0.48- | (0.37- | (0.58- | (1.10- | (0.94- | (0.79- | (1.21- |
| | | | 0.65) ** | 0.68) | 0.59) | 0.75)*** | 1.48) ** | 1.62) | 1.38) | 2.00) *** |
| CAR(12:0) / | Yes | B | 0.60 | 0.57 | 0.54 | 0.66 | 1.15 | 1.10 | 1.02 | 1.30 |
| FA(16:0) | | | (0.54- | (0.48- | (0.43- | (0.57- | (1.07- | (0.95- | (0.89- | (1.12- |
| | | | 0.65) *** | 0.66) | 0.65) | 0.74)*** | 1.32) ** | 1.38) | 1.32) | 1.58) ** |
| Met / | Yes | B | 0.57 | 0.53 | 0.54 | 0.66 | 1.12 | 1.02 | 1.15 | 1.21 |
| PC(18:1/18:1) | | | (0.51- | (0.42- | (0.44- | (0.56- | (1.02- | (0.91- | (0.93- | (1.08- |
| | | | 0.63) * | 0.64) | 0.64) | 0.75) *** | 1.17) * | 1.19) | 1.19) | 1.35) ** |
| Ala / Gln | No | B | 0.62 | 0.52 | 0.61 | 0.66 | 1.09 | 1.04 | 1.07 | 1.11 |
| | | | (0.56- | (0.40- | (0.52- | (0.57- | (1.05- | (0.92- | (1.00- | (1.05- |
| | | | 0.67) **** | 0.63) | 0.69)* | 0.74) *** | 1.15)**** | 1.12) | 1.16)* | 1.21) ** |
| SDMA / Gln | No | B | 0.59 | 0.49 | 0.54 | 0.66 | 1.06 | 1.00 | 1.03 | 1.16 |
| | | | (0.53- | (0.39- | (0.43- | (0.57- | (1.03- | (0.93- | (0.94- | (1.05- |
| | | | 0.65) ** | 0.59) | 0.66) | 0.74) *** | 1.13) ** | 1.09) | 1.15) | 1.23) ** |
| CAR(18:0) / | Yes | B | 0.60 | 0.45 | 0.54 | 0.66 | 1.17 | 0.96 | 1.13 | 1.23 |
| PC(18:1/18:1) | | | (0.54- | (0.35- | (0.43- | (0.58- | (1.05- | (0.93- | (0.91- | (1.08- |
| | | | 0.65) *** | 0.55) | 0.66) | 0.73)**** | 1.21) ** | 1.23) | 1.21) | 1.34) ** |
| Arg / | Yes | B | 0.58 | 0.55 | 0.56 | 0.65 | 1.11 | 1.04 | 1.01 | 1.22 |
| PC(18:1/18:1) | | | (0.53- | (0.44- | (0.46- | (0.56- | (1.03- | (0.94- | (0.95- | (1.08- |
| | | | 0.64) ** | 0.65) | 0.66) | 0.75) ** | 1.19) ** | 1.21) | 1.21) | 1.38) ** |
| ADMA / | Yes | C | 0.60 | 0.57 | 0.59 | 0.64 | 1.12 | 1.01 | 1.15 | 1.18 |
| PC(18:1/18:1) | | | (0.54- | (0.48- | (0.50- | (0.55- | (1.04- | (0.97- | (0.99- | (1.05- |
| | | | 0.65) *** | 0.67) | 0.68) | 0.74) ** | 1.17) *** | 1.18) | 1.21) | 1.29) ** |
| CAR(10:0) / | Yes | C | 0.58 | 0.57 | 0.49 | 0.64 | 1.25 | 1.17 | 0.92 | 1.40 |
| PC(18:1/18:1) | | | (0.52- | (0.47- | (0.38- | (0.55- | (1.07- | (0.90- | (0.74- | (1.14- |
| | | | 0.63) ** | 0.67) | 0.60) | 0.72) ** | 1.51) ** | 1.69) | 1.43) | 2.03) ** |
| 2HBA / Gln | Yes | C | 0.60 | 0.56 | 0.59 | 0.64 | 1.23 | 1.18 | 1.32 | 1.24 |
| | | | (0.54- | (0.46- | (0.47- | (0.55- | (1.07- | (0.94- | (0.98- | (1.06- |
| | | | 0.66) *** | 0.67) | 0.70) | 0.72) *** | 1.29) *** | 1.33) | 1.42) | 1.40) ** |

### Preterm Preeclampsia Prediction Using Metabolites in Pregnancy Population Strata: “White” and “Black”

In keeping with the inventors understanding that the epidemiologically observed differences in preeclampsia rates in pregnant women across different racial origins¹⁷ the existence of different underlying dominant maternal risk profile in pregnant women with different racial origin, the existence of blood biomarkers which associate particularly strong with preeclampsia risk in women with a certain racial origin and not women in women with another racial origin can be imagined. Therefore, the inventors looked for metabolites and metabolites ratios which associate strongly with preterm preeclampsia in two mutually exclusive strata of pregnant women, i.e., pregnant women which identify as from white racial origin, or in women from black racial origin, whereby the latter aggregate pregnant women whose parents were one of the Caribbean countries, Nigeria or Ghana and other African countries. In recognition that in the study population “maternal race white” make up the largest fraction of cases in the BMI<25(58%), some improved discrimination, expressed in AUROC, over the BMI<25 pregnancy population stratum was also required to qualify metabolites and metabolite ratios as specific predictors for preterm preeclampsia in the maternal race white stratum. Likewise, in the study population “maternal race black” make up the largest fraction of cases in the BMI≥30 (60%), some improved discrimination, expressed in AUROC, over the BMI≥30 pregnancy population stratum was also required to qualify metabolites and metabolite ratios as specific predictors for preterm preeclampsia in the maternal race black stratum.

### TABLE 9: Single Metabolites as Preterm Preeclampsia Predictors in Pregnancy Population Stratum: “Maternal Race Black”

To elicit the existence of metabolite biomarkers which are specifically associating with preterm preeclampsia risk in the pregnancy population stratum “maternal race Black”, we required for any given metabolite that the preterm preeclampsia risk prediction, as estimated by AUROC, was at least 0.07 AUROC units (7%) higher in the maternal race Black stratum compared to the AUROC for the same metabolite in the maternal race white stratum as well as at least 0.04 AUROC units (4%) higher than in the BMI≥30 stratum. Given the aim to discover more effective and robust parameters for assessing preeclampsia risk, we required the metabolite ratios to deliver better preterm preeclampsia risk prediction in the “maternal race Black” stratum, as estimated by AUROC, than the best single markers in TABLE 3A-PTPE therefore only metabolite ratios with AUROC>0.6 were considered.

In TABLE 9 the single metabolite is reported which, upon correction of any confounding associations, has preterm preeclampsia prediction performance, as evaluated by the statistic AUROC, which is specific to the “maternal race Black” stratum. It is found that Erg blood levels associate with preterm preeclampsia risk mostly in women of the “maternal race Black” stratum, whereby increased Erg blood levels increase the risk of preterm preeclampsia later in pregnancy.

TABLE 9
Prediction of Preterm preeclampsia in “Maternal Race Black” pregnant
women with metabolites; AUROC and Median Fold Changes Cases / Controls

| | Correction | | | | | Fold Change | | | |
|---|---|---|---|---|---|---|---|---|---|
| | for | AUROC (95% CI) | | | | Cases over Controls (95% CI) | | | |
| | confounding | | | Maternal Race | Maternal Race | | | Maternal Race | Maternal Race |
| Metabolite | (MoM) | All | BMI >= 30 | Black | White | All | BMI >= 30 | Black | White |
| Erg | Yes | 0.57 | 0.58 | 0.62 | 0.54 | 1.16 | 1.24 | 1.19 | 1.15 |
| | | (0.51-0.62) * | (0.49-0.68) | (0.54-0.70) ** | (0.45-0.63) | (1.02-1.33) * | (0.98-1.47) | (1.07-1.49) ** | (0.90-1.38) |

### TABLE 10: Metabolite Ratios as Preterm Preeclampsia Predictors in Pregnancy Population Stratum: “Maternal Race Black”

Metabolite ratios were assessed as preterm preeclampsia predictors specific to pregnant women in the “Maternal Race Black” stratum as well. Again, we required for any given metabolite ratio that the preterm preeclampsia risk prediction, as estimated by AUROC, was at least 0.07 AUROC units (7%) higher in the maternal race Black stratum compared to the AUROC for the same metabolite ratio in the maternal race white stratum as well as at least 0.04 AUROC units (4%) higher than in the BMI≥30 stratum. Given the aim to discover more effective and robust parameters for assessing preeclampsia risk, we required the metabolite ratios to deliver better preterm preeclampsia risk prediction in the “Maternal Race Black” stratum, as estimated by AUROC, than the best single marker Erg in TABLE 9 when Erg is part of the ratio OR when the metabolite ratio delivered an AUROC>0.6 together with a significance level of ** or better. A single metabolite ratio was found, constituting Erg and Cot.

TABLE 10
Prediction of Preterm preeclampsia in “Maternal Race Black” pregnant
women with metabolite ratios; AUROC and Median Fold Changes Cases / Controls

| | Correction | | | | | Fold Change | | | |
|---|---|---|---|---|---|---|---|---|---|
| | for | AUROC (95% CI) | | | | Cases over Controls (95% CI) | | | |
| Metabolite / | confounding | | | Maternal Race | Maternal Race | | | Maternal Race | Maternal Race |
| Ratio | (MoM) | All | BMI >= 30 | Black | White | All | BMI >= 30 | Black | White |
| COT / Erg | Yes | 0.55 | 0.57 | 0.64 | 0.57 | 0.84 | 0.76 | 0.71 | 0.84 |
| | | (0.50- | (0.47-0.67) | (0.56- | (0.48-0.67) | (0.70- | (0.60-1.08) | (0.55- | (0.58-1.04) |
| | | 0.61) | | 0.71) *** | | 1.02) | | 0.87) ** | |

### TABLE 11: Single Metabolites as Preterm Preeclampsia Predictors in Pregnancy Population Stratum: “Maternal Race White”

To elicit the existence of metabolite biomarkers which are specifically associating with preterm preeclampsia risk in the pregnancy population stratum “maternal race White”, we required for any given metabolite that the preterm preeclampsia risk prediction, as estimated by AUROC, was at least 0.07 AUROC units (7%) higher in the maternal race White stratum compared to the AUROC for the same metabolite in the maternal race Black stratum as well as at least 0.04 AUROC units (4%) higher than in the BMI<25 stratum. Given the aim to discover more effective and robust parameters for assessing preeclampsia risk, we required the metabolite ratios to deliver better preterm preeclampsia risk prediction in the “maternal race White” stratum, as estimated by AUROC, than the best single markers in TABLE 3A-PTPE therefore only metabolite ratios with AUROC>0.6 were considered. In TABLE 11 the metabolites are reported which, upon correction of any confounding associations, have preterm preeclampsia prediction performance, as evaluated by the statistic AUROC, which is specific to the “maternal race White” stratum. It is found that 25(OH)D3 and LPC(16:0) blood levels associate more strongly with preterm preeclampsia risk mostly in women of the “maternal race White” stratum, whereby increased blood levels of 25(OH)D3 and/or LPC(16:0) increase the risk of preterm preeclampsia later in pregnancy.

TABLE 11
Prediction of Preterm preeclampsia in “Maternal Race White” pregnant
women with metabolites; AUROC and Median Fold Changes Cases / Controls

| | Correction | | | | | Fold Change | | | |
|---|---|---|---|---|---|---|---|---|---|
| | for | AUROC (95% CI) | | | | Cases over Controls (95% CI) | | | |
| | confounding | | | Maternal Race | Maternal Race | | | Maternal Race | Maternal Race |
| Metabolite | (MoM) | All | BMI < 25 | Black | White | All | BMI < 25 | Black | White |
| 25(OH)D3 | Yes | 0.60 | 0.59 | 0.55 | 0.63 | 1.16 | 1.08 | 1.13 | 1.18 |
| | | (0.54- | (0.48- | (0.47- | (0.54- | (1.07- | (0.98- | (0.95- | (1.06- |
| | | 0.66) *** | 0.69) | 0.64) | 0.71) ** | 1.26) *** | 1.30) | 1.26) | 1.31) ** |
| LPC(16:0) | Yes | 0.55 | 0.53 | 0.52 | 0.63 | 1.01 | 1.02 | 0.96 | 1.06 |
| | | (0.49- | (0.43- | (0.43- | (0.55- | (1.00- | (0.97- | (0.94- | (1.02- |
| | | 0.61) | 0.63) | 0.61) | 0.71) ** | 1.06) | 1.07) | 1.04) | 1.11) ** |

### TABLE 12: Metabolite Ratios as Preterm Preeclampsia Predictors in Pregnancy Population Stratum: “Maternal Race White”

Metabolite ratios were assessed as preterm preeclampsia predictors specific to pregnant women in the “Maternal Race White” stratum as well. Again, we required for any given metabolite ratio that the preterm preeclampsia risk prediction, as estimated by AUROC, was at least 0.07 AUROC units (7%) higher in the maternal race White stratum compared to the AUROC for the same metabolite ratio in the maternal race Black stratum as well as at least 0.04 AUROC units (4%) higher than in the BMI<25 stratum. Given the aim to discover more effective and robust parameters for assessing preeclampsia risk, we required the metabolite ratios to deliver better preterm preeclampsia risk prediction in the “Maternal Race Black” stratum, as estimated by AUROC, than the best single markers 25(OH)D3 or LPC(16:0) in TABLE 11 when these metabolites are part of the ratio (Group A) OR when the metabolite ratio delivered an AUROC>0.6 together with a significance level of ** or better (Group B).

In TABLE 12, the metabolite ratios are reported which, upon correction of any confounding associations, have preterm preeclampsia prediction performance, as evaluated by the statistic AUROC, which is specific to the “Maternal Race White” stratum. From Table 12, it is apparent that the ratio of metabolite blood levels constituting LPC(16:0) and Gln further improves the specific preterm preeclampsia prediction of the single maker LPC(16:0) (TABLE 11) in the Maternal Race White; no metabolite ratios improving on 25(OH)D3 were identified. Interestingly, all but one other metabolite ratios with associate strongly with preterm preeclampsia risk in the “Maternal Race White” stratum constitute 2HBA, and any of 3HBA, CAR (2:0), FA (14:0), FA (16:0), FA (20:3n6), FA (20:4n6), FA (20:5n3), LA or Leu. In addition, a metabolite ratio constituting Met and 2H(2/3) MBA also associates more strongly with preterm preeclampsia risk in pregnant women in the pregnancy population stratum “maternal race white”.

TABLE 12
Prediction of Preterm preeclampsia in “Maternal Race White” pregnant
women with metabolite ratios; AUROC and Median Fold Changes Cases / Controls

| | | | | | | | Fold Change | | | |
|---|---|---|---|---|---|---|---|---|---|---|
| | Correction | | AUROC (95% CI) | | | | Cases over Controls (95% CI) | | | |
| | for | | | | Maternal | Maternal | | | Maternal | Maternal |
| Metabolite | confounding | | | | Race | Race | | | Race | Race |
| Ratio | (MoM) | Group | All | BMI < 25 | Black | White | All | BMI < 25 | Black | White |
| 2HBA / | Yes | A | 0.56 | 0.55 | 0.50 | 0.65 | 1.05 | 1.11 | 0.93 | 1.31 |
| FA(20:3n6) | | | (0.50- | (0.44- | (0.41- | (0.58- | (1.01- | (0.90- | (0.85- | (1.13- |
| | | | 0.62) * | 0.65) | 0.59) | 0.72) **** | 1.26) * | 1.31) | 1.21) | 1.51) *** |
| 2HBA / | No | A | 0.60 | 0.55 | 0.57 | 0.64 | 1.16 | 1.02 | 1.12 | 1.33 |
| FA(16:0) | | | (0.54- | (0.43- | (0.48- | (0.56- | (1.07- | (0.92- | (0.98- | (1.11- |
| | | | 0.66) ** | 0.66) | 0.66) | 0.73) *** | 1.29) *** | 1.28) | 1.28) | 1.45) *** |
| LPC(16:0) / | Yes | A | 0.59 | 0.52 | 0.55 | 0.64 | 1.07 | 1.05 | 1.05 | 1.10 |
| Gln | | | (0.53- | (0.41- | (0.46- | (0.57- | (1.02- | (0.94- | (0.97- | (1.05- |
| | | | 0.64) ** | 0.64) | 0.64) | 0.72) *** | 1.12) ** | 1.11) | 1.12) | 1.19) *** |
| 2HBA / Leu | No | B | 0.58 | 0.57 | 0.56 | 0.63 | 1.10 | 1.07 | 1.06 | 1.18 |
| | | | (0.53- | (0.46- | (0.48- | (0.54- | (1.04- | (0.94- | (0.96- | (1.08- |
| | | | 0.64) ** | 0.68) | 0.63) | 0.71) ** | 1.26) ** | 1.38) | 1.22) | 1.45) ** |
| 2HBA / | Yes | B | 0.58 | 0.53 | 0.56 | 0.63 | 1.15 | 1.01 | 1.10 | 1.26 |
| FA(14:0) | | | (0.53- | (0.43- | (0.47- | (0.55- | (1.05- | (0.88- | (0.96- | (1.09- |
| | | | 0.64) ** | 0.64) | 0.65) | 0.71) ** | 1.30) ** | 1.27) | 1.32) | 1.46) ** |
| 2HBA / | No | B | 0.57 | 0.53 | 0.54 | 0.63 | 1.12 | 0.93 | 1.12 | 1.27 |
| FA(20:4n6) | | | (0.51- | (0.41- | (0.45- | (0.54- | (1.02- | (0.80- | (0.93- | (1.08- |
| | | | 0.63) * | 0.64) | 0.63) | 0.71) ** | 1.23) * | 1.14) | 1.22) | 1.43) ** |
| 2HBA / | Yes | B | 0.58 | 0.56 | 0.53 | 0.63 | 1.12 | 1.05 | 1.04 | 1.29 |
| CAR(2:0) | | | (0.52- | (0.45- | (0.45- | (0.55- | (1.03- | (0.94- | (0.93- | (1.08- |
| | | | 0.64) ** | 0.66) | 0.62) | 0.72) ** | 1.22) ** | 1.27) | 1.18) | 1.38) ** |
| Met / | Yes | B | 0.57 | 0.58 | 0.55 | 0.62 | 0.90 | 0.89 | 0.96 | 0.83 |
| 2H(2/3)MBA | | | (0.52- | (0.47- | (0.47- | (0.54- | (0.82- | (0.73- | (0.78- | (0.76- |
| | | | 0.63) ** | 0.69) | 0.63) | 0.69) ** | 0.97) * | 1.03) | 1.06) | 0.96) ** |
| 2HBA / | No | B | 0.55 | 0.52 | 0.53 | 0.62 | 1.18 | 1.07 | 0.97 | 1.30 |
| 3HBA | | | (0.50- | (0.42- | (0.44- | (0.54- | (1.00- | (0.84- | (0.77- | (1.08- |
| | | | 0.61) | 0.62) | 0.61) | 0.70) ** | 1.26) | 1.28) | 1.14) | 1.50) ** |
| 2HBA / | Yes | B | 0.54 | 0.53 | 0.52 | 0.61 | 1.07 | 1.06 | 0.89 | 1.22 |
| FA(20:5n3) | | | (0.48- | (0.42- | (0.43- | (0.54- | (0.95- | (0.83- | (0.75- | (1.07- |
| | | | 0.60) | 0.65) | 0.61) | 0.69) ** | 1.27) | 1.44) | 1.20) | 1.58) ** |
| 2HBA / LA | Yes | B | 0.56 | 0.53 | 0.49 | 0.61 | 1.11 | 1.05 | 0.94 | 1.19 |
| | | | (0.50- | (0.44- | (0.41- | (0.53- | (1.00- | (0.88- | (0.89- | (1.04- |
| | | | 0.61) * | 0.62) | 0.57) | 0.69) ** | 1.22) * | 1.22) | 1.16) | 1.40) * |

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A method of early prediction of risk of pre-term pre-eclampsia in a pregnant human subject having a BMI of less than 25, the method comprising the steps of:
providing an abundance of ornithine in a biological sample obtained from the subject at an early stage of pregnancy;
comparing the abundance of ornithine in the subject to a reference abundance; and
providing an estimation of the risk of pre-term pre-eclampsia in the subject based on the comparison.

2. A method according to claim 1, in which the reference abundance is a reference abundance of ornithine.

3. A method according to claim 2, in which:
the reference abundance of ornithine is the abundance of ornithine in a negative control, wherein a reduced abundance of ornithine relative to the reference abundance correlates with risk of pre-term pre-eclampsia; or
the reference abundance or ornithine is the abundance of ornithine in a positive control, wherein a similar abundance of ornithine relative to the reference abundance correlates with risk of pre-term pre-eclampsia.

4. A method according to claim 1, in which:
the reference abundance is an abundance of a second metabolite biomarker in a biological sample obtained from the subject at an early stage of pregnancy, in which the second metabolite biomarker is selected from threonine, Asymmetric dimethylarginine (ADMA), 1-Oleoyl-2-hydroxy-sn-glycero-3-phosphocholine, 1-Palmitoyl-2-hydroxy-sn-glycero-3-phosphocholine, alanine and glutamine; and
the comparing step comprises calculating a ratio of the abundance of ornithine and the abundance of one of the second metabolite biomarkers.

5. A method according to claim 4, in which:
the ratio is second metabolite biomarker/ornithine; and
the second metabolite biomarker is selected from threonine, Asymmetric dimethylarginine (ADMA), 1-Oleoyl-2-hydroxy-sn-glycero-3-phosphocholine, 1-Palmitoyl-2-hydroxy-sn-glycero-3-phosphocholine, alanine,
wherein a ratio of greater than 1 (for example at least 1.1, 1.5 or 1.2) correlates with risk of pre-term pre-eclampsia.

6. A method according to claim 4, in which:
the ratio is second metabolite biomarker/ornithine; and
the second metabolite biomarker is glutamine,
wherein a ratio of less than 1 (for example less than 0.95 or 0.9) correlates with risk of pre-term pre-eclampsia.

7. A method of early prediction of risk of pre-term pre-eclampsia in a pregnant human subject having a BMI of 30 or greater, the method comprising the steps of:
providing an abundance of a metabolite biomarker in a biological sample obtained from the subject at an early stage of pregnancy;
comparing the abundance of the biomarker in the subject to a reference abundance; and
providing an estimation of the risk of pre-term pre-eclampsia in the subject based on the comparison,
in which the metabolite biomarker is selected from 2-Hydroxy-(2/3)-methylbutyric acid, Dodecanoylcarnitine, Alanine, Decanoylcarnitine and Symmetric dimethylarginine.

8. A method according to claim 7, in which the reference abundance is a reference abundance of the metabolite biomarker.

9. A method according to claim 8, in which:
the reference abundance of the metabolite biomarker is the abundance of the metabolite biomarker in a negative control, wherein an increased abundance of the metabolite biomarker relative to the reference abundance correlates with risk of pre-term pre-eclampsia; or
the reference abundance of the metabolite biomarker is the abundance of the metabolite biomarker in a positive control, wherein a similar abundance of the metabolite biomarker relative to the reference abundance correlates with risk of pre-term pre-eclampsia.

10. A method according to claim 7, in which:
the metabolite biomarker is 2-Hydroxy-(2/3)-methylbutyric acid;
the reference abundance is an abundance of a second metabolite biomarker in a biological sample obtained from the subject at an early stage of pregnancy, in which the second metabolite is selected from 1-(1Z-Octadecenyl)-2-oleoyl-sn-glycero-3-phosphocholine and glutamine; and
the comparing step comprises calculating a ratio of the abundance of 2-Hydroxy-(2/3)-methylbutyric acid and the abundance of one of the second metabolite biomarkers.

11. A method according to claim 10, in which:
the ratio is selected from:
2-Hydroxy-(2/3)-methylbutyric acid/1-(1Z-Octadecenyl)-2-oleoyl-sn-glycero-3-phosphocholine; and
2-Hydroxy-(2/3)-methylbutyric acid/glutamine,
wherein a ratio of greater than 1 (for example at least 1.2 or 1.3) correlates with risk of pre-term pre-eclampsia.

12. A method according to claim 7 in which:
the metabolite biomarker is 2-Hydroxybutyric acid; and
the comparing step comprises calculating a ratio of the abundance of 2-Hydroxybutyric acid and the abundance of 1-(1Z-Octadecenyl)-2-oleoyl-sn-glycero-3-phosphocholine.

13. A method according to claim 12, in which:
the ratio is 2-Hydroxybutyric acid/1-(1Z-Octadecenyl)-2-oleoyl-sn-glycero-3-phosphocholine; and
wherein a ratio of greater than 1 (for example at least 1.1 or 1.2) correlates with risk of pre-term pre-eclampsia.

14. A method according to claim 7 in which:
the metabolite biomarker is Dodecanoylcarnitine; and
the comparing step comprises calculating a ratio of the abundance of Dodecanoylcarnitine and the abundance of Myristic acid.

15. A method according to claim 14, in which:
the ratio is Dodecanoylcarnitine/Myristic acid; and
wherein a ratio of greater than 1 (for example at least 1.2.1.3 or 1.4) correlates with risk of pre-term pre-eclampsia.

16. A method of early prediction of risk of pre-term pre-eclampsia in a pregnant human subject of white racial origin, the method comprising the steps of:
providing an abundance of a metabolite biomarker selected from 25-Hydroxyvitamin D3 or 1-Palmitoyl-2-hydroxy-sn-glycero-3-phosphocholine in a biological sample obtained from the subject at an early stage of pregnancy;
comparing the abundance of the metabolite biomarker in the subject to a reference abundance of the same metabolite biomarker; and
providing an estimation of the risk of pre-term pre-eclampsia in the subject based on the comparison.

17. A method according to claim 16, in which:
an increased abundance of the metabolite biomarker relative to the reference abundance of the metabolite biomarker in a negative control correlates with risk of pre-term pre-eclampsia; or
a similar abundance of the metabolite biomarker relative to the reference abundance of the metabolite biomarker in a positive control correlates with risk of pre-term pre-eclampsia.

18. A method of early prediction of risk of pre-term pre-eclampsia in a pregnant human subject of white racial origin, the method comprising the steps of:
providing an abundance of a plurality of biomarkers from Table 12 in a biological sample obtained from the subject at an early stage of pregnancy the plurality of biomarkers including a first metabolite biomarker selected from 2-Hydroxybutyric acid, 1-Palmitoyl-2-hydroxy-sn-glycero-3-phosphocholine and methionine,
and a second biomarker selected from
8,11,14 Eicosatrienoic acid, Palmitic acid, Glutamine, Leucine, Myristic acid, Arachidonic acid, Acetylcarnitine, 2-Hydroxy-(2/3)-methylbutyric acid, 3-Hydroxybutyric acid, Eicosapentaenoic acid, and Lactic acid;
calculating a ratio of a first metabolite biomarker to a second metabolite biomarker; and
providing an estimation of the risk of pre-term pre-eclampsia in the subject based on the comparison.

19. A method according to claim 18, in which the ratio of the first metabolite biomarker to the second metabolite biomarker is a metabolite ratio of Table 12.

20. A system to calculate risk of pre-term pre-eclampsia in a pregnant human subject having a BMI of less than 25, the system a processor configured to:
receive as an input an abundance of ornithine in a biological sample obtained from the subject at an early stage of pregnancy;
compare the abundance of ornithine in the subject to a reference abundance;
calculate the risk of pre-term pre-eclampsia in the subject based on the comparison; and
provide an output of the risk.

21. A system according to claim 20, in which the reference abundance is a reference abundance of ornithine.

22. A system according to claim 21, in which:
the reference abundance of ornithine is the abundance of ornithine in a negative control, wherein a reduced abundance of ornithine relative to the reference abundance correlates with risk of pre-term pre-eclampsia; or
the reference abundance or ornithine is the abundance of ornithine in a positive control, wherein a similar abundance of ornithine relative to the reference abundance correlates with risk of pre-term pre-eclampsia.

23. A system according to claim 20, in which:
the reference abundance is an abundance of a second metabolite biomarker in a biological sample obtained from the subject at an early stage of pregnancy, in which the second metabolite biomarker is selected from threonine, Asymmetric dimethylarginine (ADMA), 1-Oleoyl-2-hydroxy-sn-glycero-3-phosphocholine, 1-Palmitoyl-2-hydroxy-sn-glycero-3-phosphocholine, alanine and glutamine; and
the comparing step comprises calculating a ratio of the abundance of ornithine and the abundance of one of the second metabolite biomarkers.

24. A system according to claim 23, in which:
the ratio is second metabolite biomarker/ornithine; and
the second metabolite biomarker is selected from threonine, Asymmetric dimethylarginine (ADMA), 1-Oleoyl-2-hydroxy-sn-glycero-3-phosphocholine, 1-Palmitoyl-2-hydroxy-sn-glycero-3-phosphocholine, alanine,
wherein a ratio of greater than 1 (for example at least 1.1, 1.5 or 1.2) correlates with risk of pre-term pre-eclampsia.

25. A system according to claim 23, in which:
the ratio is second metabolite biomarker/ornithine; and
the second metabolite biomarker is glutamine,
wherein a ratio of less than 1 (for example less than 0.95 or 0.9) correlates with risk of pre-term pre-eclampsia.

26. A system to calculate risk of pre-term pre-eclampsia in a pregnant human subject having a BMI of 30 or greater, the system comprising a processor configured to:
receive as an input an abundance of a metabolite biomarker in a biological sample obtained from the subject at an early stage of pregnancy;
compare the abundance of the biomarker in the subject to a reference abundance;
calculate the risk of pre-term pre-eclampsia in the subject based on the comparison; and
provide an output of the risk,
in which the metabolite biomarker is selected from 2-Hydroxy-(2/3)-methylbutyric acid, Dodecanoylcarnitine, Alanine, Decanoylcarnitine and Symmetric dimethylarginine.

27. A system according to claim 26, in which the reference abundance is a reference abundance of the metabolite biomarker.

28. A system according to claim 27, in which:
the reference abundance of the metabolite biomarker is the abundance of the metabolite biomarker in a negative control, wherein an increased abundance of the metabolite biomarker relative to the reference abundance correlates with risk of pre-term pre-eclampsia; or
the reference abundance of the metabolite biomarker is the abundance of the metabolite biomarker in a positive control, wherein a similar abundance of the metabolite biomarker relative to the reference abundance correlates with risk of pre-term pre-eclampsia.

29. A system according to claim 26, in which:
the metabolite biomarker is 2-Hydroxy-(2/3)-methylbutyric acid;
the reference abundance is an abundance of a second metabolite biomarker in a biological sample obtained from the subject at an early stage of pregnancy, in which the second metabolite is selected from 1-(1Z-Octadecenyl)-2-oleoyl-sn-glycero-3-phosphocholine and glutamine; and
the comparing step comprises calculating a ratio of the abundance of 2-Hydroxy-(2/3)-methylbutyric acid and the abundance of one of the second metabolite biomarkers.

30. A system according to claim 29, in which:
the ratio is selected from:
2-Hydroxy-(2/3)-methylbutyric acid/1-(1Z-Octadecenyl)-2-oleoyl-sn-glycero-3-phosphocholine; and
2-Hydroxy-(2/3)-methylbutyric acid/glutamine,
wherein a ratio of greater than 1 (for example at least 1.2 or 1.3) correlates with risk of pre-term pre-eclampsia.

31. A system according to claim 26 in which:
the metabolite biomarker is 2-Hydroxybutyric acid; and
the comparing step comprises calculating a ratio of the abundance of 2-Hydroxybutyric acid and the abundance of 1-(1Z-Octadecenyl)-2-oleoyl-sn-glycero-3-phosphocholine.

32. A system according to claim 31, in which:
the ratio is 2-Hydroxybutyric acid/1-(1Z-Octadecenyl)-2-oleoyl-sn-glycero-3-phosphocholine; and
wherein a ratio of greater than 1 (for example at least 1.1 or 1.2) correlates with risk of pre-term pre-eclampsia.

33. A system according to claim 26 in which:
the metabolite biomarker is Dodecanoylcarnitine; and
the comparing step comprises calculating a ratio of the abundance of Dodecanoylcarnitine and the abundance of Myristic acid.

34. A method according to claim 33, in which:
the ratio is Dodecanoylcarnitine/Myristic acid; and
wherein a ratio of greater than 1 (for example at least 1.2.1.3 or 1.4) correlates with risk of pre-term pre-eclampsia.

35. A system to calculate risk of pre-term pre-eclampsia in a pregnant human subject of white racial origin, the system comprising a processor configured to:
receive as an input an abundance of a metabolite biomarker selected from 25-Hydroxyvitamin D3 or 1-Palmitoyl-2-hydroxy-sn-glycero-3-phosphocholine in a biological sample obtained from the subject at an early stage of pregnancy;
compare the abundance of the metabolite biomarker in the subject to a reference abundance of the same metabolite biomarker;
calculate the risk of pre-term pre-eclampsia in the subject based on the comparison; and
provide an output of the risk.

36. A method according to claim 35, in which:
an increased abundance of the metabolite biomarker relative to the reference abundance of the metabolite biomarker in a negative control correlates with risk of pre-term pre-eclampsia; or
a similar abundance of the metabolite biomarker relative to the reference abundance of the metabolite biomarker in a positive control correlates with risk of pre-term pre-eclampsia.

37. A system to calculate risk of pre-term pre-eclampsia in a pregnant human subject of white racial origin, the system comprising a processor configured to:
Receive as an input an abundance of a plurality of biomarkers from Table 12 in a biological sample obtained from the subject at an early stage of pregnancy the plurality of biomarkers including a first metabolite biomarker selected from 2-Hydroxybutyric acid, 1-Palmitoyl-2-hydroxy-sn-glycero-3-phosphocholine and methionine,
and a second biomarker selected from
8,11,14 Eicosatrienoic acid, Palmitic acid, Glutamine, Leucine, Myristic acid, Arachidonic acid, Acetylcarnitine, 2-Hydroxy-(2/3)-methylbutyric acid, 3-Hydroxybutyric acid, Eicosapentaenoic acid, and Lactic acid;
calculate a ratio of a first metabolite biomarker to a second metabolite biomarker;
calculate the risk of pre-term pre-eclampsia in the subject based on the calculated ratio of the first and second metabolite biomarkers; and
provide an output of the risk.

38. A system according to claim 37, in which the ratio is calculated based on the first metabolite biomarker and the second metabolite biomarker pairs of Table 12.
